(19) United States
(12) Patent Application Publication
(10) Pub. No.: US 2025/0327132 A1
(43) Pub. Date: Oct. 23, 2025

(54) Lung Cancer-Related Biomarkers and Methods of Using the Same

(71) Applicants: The Government of the United States, as Represented by the Director of the Defense Health Agency, Fort Detrick, MD (US); The Henry M. Jackson Foundation for the Advancement of Military Medicine, Inc., Bethesda, MD (US)

(72) Inventors: Matthew Wilkerson, Bethesda, MD (US); Robert Browning, Bethesda, MD (US); Craig Shriver, Bethesda, MD (US)

(21) Appl. No.: 18/871,197

(22) PCT Filed: Jun. 13, 2023
(86) PCT No.: PCT/US2023/025161

§ 371 Date: Dec. 3, 2024

Related U.S. Application Data
(60) Provisional application No. 63/351,686, filed on Jun. 13, 2022.

(51) Int. Cl. C12Q 1/6886; C12Q 1/6874; G01N 33/574; G01N 33/68; G16B 20/00

(52) U.S. Cl. CPC C12Q 1/6886; C12Q 1/6874; G01N 33/57488; G01N 33/6848; G16B 20/00; C12Q 2600/112; C12Q 2600/156; C12Q 2600/158

(57) ABSTRACT

This application relates generally to lung cancer-related biomarkers, such as aggressive lung cancer-related molecules, which can be used to predict clinical outcomes, such as patient overall survival and/or metastasis-free survival, and methods of using the same to diagnose, prognose, monitor, and treat lung cancer, such as lung adenocarcinoma, in a subject

### CROSS-REFERENCE TO RELATED APPLICATIONS

This application claims priority to U.S. Provisional Application No. 63/351,686 filed 13 Jun. 2022, the entire contents of which are hereby incorporated by reference in their entirety.

### STATEMENT REGARDING FEDERALLY SPONSORED RESEARCH

This invention was made with government support under HU0001-21-2-0002, HU0001-18-2-0032, HU0001-14-2-0041 and HU0001-18-2-0038 awarded by the Uniformed Services University of the Health Sciences. The government has certain rights in the invention.

### FIELD

This application relates generally to lung cancer-related biomarkers, such as aggressive lung cancer-related molecules, which can be used to predict clinical outcomes, such as patient overall survival and/or metastasis-free survival, and methods of using the same to diagnose, prognose, monitor, and treat lung cancer, such as lung adenocarcinoma, in a subject.

### BACKGROUND

Lung cancer, also known as lung carcinoma, is a malignant tumor that begins in the lung. There are two main types of lung cancer based on the type of cells the tumor is derived from: small cell lung cancer (SCLC; about 15% of cases) and non-small-cell lung cancer (NSCLC; about 85% of cases). NSCLCs comprise a group of three cancer types: adenocarcinoma, squamous-cell carcinoma, and large-cell carcinoma. Nearly 40% of lung cancers are adenocarcinomas.

Lung adenocarcinoma (LUAD) is a leading cause of cancer deaths in the United States despite advances in therapeutics targeting somatically-altered genes and immune checkpoints. A major challenge in diagnosing and treating individuals with LUAD is the vast morphological and molecular heterogeneity within and among tumors. Several national and international molecular profiling efforts have cataloged a diversity of somatic DNA alterations in LUAD, including driver gene mutations, copy number alterations, fusion genes, as well as molecular subtypes defined by RNA expression. Despite these advances, however, it remains challenging to predict clinical outcomes for individuals with LUAD based on clinical or molecular characteristics. In addition, many LUAD tumors do not possess a molecular alteration currently indicated for targeted therapy.

Thus, there remains a need for improved molecular signatures of lung cancer, such as LUAD, that can be used to better diagnose, prognose, and/or monitor lung cancer, such as LUAD, in a subject, as well as to better predict treatment outcomes.

### SUMMARY

Disclosed herein are lung cancer-related biomarkers, such as aggressive lung cancer-related molecules, which can be used to predict clinical outcomes, such as patient overall survival and/or metastasis-free survival, and methods of using the same to diagnose, prognose, monitor, and treat lung cancer, such as lung adenocarcinoma (LUAD), in a subject. The present disclosure encompasses, in some aspects, the observation that the genes provided in Table 1 and Table 2, particularly those provided in Table 2, are differentially regulated (e.g., up-regulated or down-regulated) in LUAD and the aggregate expression of some or all of the genes provided in Table 1, particularly those provided in Table 2, can discriminate patients with LUAD by overall survival and metastasis-free survival.

Accordingly, in one aspect, provided herein is a method of identifying the risk of a worsening prognosis of lung cancer in a subject in need thereof, the method comprising: a) measuring relative expression levels of a plurality of aggressive lung cancer-related molecules in a tumor sample from the subject, wherein the plurality of aggressive lung cancer-related molecules is selected from Table 1; b) combining the relative expression levels of the plurality of aggressive lung cancer-related molecules to generate a score representing an aggregate expression of the plurality of aggressive lung cancer-related molecules; and c) comparing the score to a reference cohort comprising a first group of subjects previously identified as having a low risk of a worsening prognosis of lung cancer and a second group of subjects previously identified as having a high risk of a worsening prognosis of lung cancer, each group having a range of reference scores associated therewith, wherein, if the score is within the range of reference scores associated with the first group, the subject is at low risk of the worsening prognosis of lung cancer, and wherein, if the score is within the range of reference scores associated with the second group, the subject is at high risk of the worsening prognosis of lung cancer. In some embodiments, the reference cohort further comprises a third group of subjects previously identified as having a medium risk of a worsening prognosis of lung cancer, said third group has a range of reference scores associated therewith, and wherein, if the score is within the range of reference scores associated with the third group, the subject is at medium risk of the worsening prognosis of lung cancer.

In some embodiments, the method further comprises administering a therapeutically effective amount of a lung cancer therapy to the subject identified as having high or medium risk of the worsening prognosis of lung cancer. In a related aspect, also provided herein is a method of treating lung cancer, the method comprising administering a therapeutically effective amount of a lung cancer therapy to the subject identified as having high or medium risk of a worsening prognosis of lung cancer, as described herein. In some embodiments, the lung cancer is LUAD and the method further comprises, prior to administering the lung cancer therapy, identifying a molecular subtype of the LUAD in the subject. In some embodiments, the lung cancer therapy comprises: a) administering a therapeutically effective amount of one or more immunotherapeutic treatments if the molecular subtype of the LUAD is a proximal-inflammatory (PI) subtype; b) administering a therapeutically effective amount of one or more inhibitory compounds targeting epidermal growth factor receptor (EGFR) signaling and/or kinase activity from protein kinase C epsilon (PRKCE) and/or ribosomal protein S6 kinase A1 (RPS6KA1) if the molecular subtype of the LUAD is a terminal respiratory unit (TRU) subtype; or c) administering a therapeutically effective amount of one or more cyclin-dependent kinase (CDK) inhibitors and/or glutaminase inhibitors if the molecular subtype of the LUAD is a proximal-proliferative (PP) subtype. In some embodiments, the PI subtype is identified by overexpression of the immune cell markers cluster of differentiation 163 (CD163) and/or vascular cell adhesion protein 1 (VCAM1), the TRU subtype is identified by overexpression of surfactant protein C (SFTPC) and/or thyroid transcription factor 1 (NKX2-1 or TTF1), and the PP subtype is identified by overexpression of thymine DNA glycosylase (TDG) and/or glutathione peroxidase 2 (GPX2).

In another aspect, provided herein is a method of predicting the risk of developing metastasis in a subject having lung cancer, the method comprising: a) measuring relative expression levels of a plurality of aggressive lung cancer-related molecules in a tumor sample from the subject, wherein the plurality of aggressive lung cancer-related molecules is selected from Table 1; b) combining the relative expression levels of the plurality of aggressive lung cancer-related molecules to generate a score representing an aggregate expression of the plurality of aggressive lung cancer-related molecules; and c) comparing the score to a reference cohort comprising a first group of subjects previously identified as having a low risk of developing metastasis and a second group of subjects previously identified as having a high risk of developing metastasis, each group having a range of reference scores associated therewith, wherein, if the score is within the range of reference scores associated with the first group, the subject is at low risk of developing metastasis, and wherein, if the score is within the range of reference scores associated with the second group, the subject is at high risk of developing metastasis. In some embodiments, the reference cohort further comprises a third group of subjects previously identified as having a medium risk of developing metastasis, said third group has a range of reference scores associated therewith, and wherein, if the score is within the range of reference scores associated with the third group, the subject is at medium risk of developing metastasis. In some embodiments, the subject has LUAD.

In some embodiments, the method further comprises administering a therapeutically effective amount of a preventive cancer therapy to the subject identified as having high or medium risk of developing metastasis. In a related aspect, also provided herein is a method of treating lung cancer, the method comprising administering a therapeutically effective amount of a preventive lung cancer therapy to a subject identified as having high or medium risk of developing metastasis, as described herein. In some embodiments, the preventive cancer therapy comprises a chemoprevention treatment.

In a further aspect, the disclosure provides a method of monitoring effectiveness of a cancer therapy in a subject having lung cancer, the method comprising: a) measuring relative expression levels of a plurality of aggressive lung cancer-related molecules in a tumor sample from the subject before and after the cancer treatment, wherein the plurality of aggressive lung cancer-related molecules is selected from Table 1; b) combining the relative expression levels of the plurality of aggressive lung cancer-related molecules before the cancer treatment to generate a pre-treatment score representing an aggregate expression of the plurality of aggressive lung cancer-related molecules before the cancer treatment and combining the relative expression levels of the plurality of aggressive lung cancer-related molecules after the cancer treatment to generate a post-treatment score representing an aggregate expression of the plurality of aggressive lung cancer-related molecules after the cancer treatment, wherein a lower post-treatment score as compared to the pre-treatment score indicates that the cancer treatment is effective. In some embodiments, the subject has LUAD. In some embodiments, the method further comprises changing the cancer treatment (e.g., administering a therapeutically effective amount of a different cancer treatment to the subject) if the post-treatment score is higher than the pre-treatment score.

In some embodiments, the plurality of aggressive lung cancer-related molecules used in any of the methods disclosed herein is selected from CLIP1, AVEN, SRPRA, PUS1, MYO1E, KIF26B, FOSL2, MATR3, RPS6KA5, TOR1AIP1, MTX3, UTRN, TMX4, and MCCC1. In some embodiments, the plurality of aggressive lung cancer-related molecules comprises CLIP1, AVEN, SRPRA, PUS1, MYO1E, KIF26B, FOSL2, MATR3, RPS6KA5, TOR1AIP1, MTX3, UTRN, TMX4, and MCCC1. In some embodiments, the plurality of aggressive lung cancer-related molecules consists of CLIP1, AVEN, SRPRA, PUS1, MYO1E, KIF26B, FOSL2, MATR3, RPS6KA5, TOR1AIP1, MTX3, UTRN, TMX4, and MCCC1. In some embodiments, the relative expression levels of the plurality of aggressive lung cancer-related molecules are measured based on protein expression and/or RNA expression. In some embodiments, the score is calculated by:

$$Score = {\sum }_{i = 1}^{k} {β}_{i} * {expression}_{i}$$

wherein k is the number of the plurality of aggressive lung cancer-related molecules, β is the coefficient assigned to aggressive lung cancer-related molecule i provided in Table 1, and expression is the expression level in the tumor sample.

In a yet another aspect, the disclosure provides a method of treating LUAD in a subject in need thereof, the method comprising: a) identifying a molecular subtype of the LUAD in the subject; and b) administering a lung cancer therapy to the subject according to the molecular subtype of the lung adenocarcinoma identified, wherein the lung cancer therapy comprises: i) administering a therapeutically effective amount of one or more immunotherapeutic treatments if the molecular subtype of the LUAD is a PI subtype; ii) administering a therapeutically effective amount of one or more inhibitory compounds targeting EGFR signaling and/or kinase activity from PRKCE and/or RPS6KA1 if the molecular subtype of the LUAD is a TRU subtype; or iii) administering a therapeutically effective amount of one or more CDK inhibitors and/or glutaminase inhibitors if the molecular subtype of the LUAD is a PP subtype. In some embodiments, the PI subtype is identified by overexpression of CD163 and/or VCAM1, the TRU subtype is identified by overexpression of SFTPC and/or NKX2-1 (or TTF1), and the PP subtype is identified by overexpression of TDG and/or GPX2.

### BRIEF DESCRIPTION OF THE DRAWINGS

The accompanying drawings, which are incorporated in and constitute a part of this specification, illustrate certain embodiments, and together with the written description, serve to explain certain principles of the compositions and methods disclosed herein.

FIG. 1A-IG depict the subtyping of lung adenocarcinoma (LUAD) by whole genome somatic signatures. FIG. 1A: Clustering of LUAD by somatic single nucleotide variant (SNV), insertion/deletion (indel) and structural variant (SV) signatures. Columns are tumors and rows are somatic signature values and patient/tumor features (tumor n=87). Patients and tumor features are tested for association with somatic signature subtype, * ANOVA (p<0.05). \hat{ }χ² test (p<0.05). FIG. 1B-1G: Transition/transversion ratios, SV deletions, SV inversions, and mutant TP53 expression scores compared across somatic signature subtypes. Boxplot lines indicates 25%, 50%, 75% percentiles, points are tumors, with horizontal jitter added for visualization. p′ refers to Wilcoxon rank sum test on structurally-altered vs transition subtype. p″ refers to Wilcoxon rank sum on structurally-altered vs transversion subtype. p refers to Wilcoxon rank sum test on structurally altered versus other subtypes.

FIG. 2A-2D depict the correlation of gene-wise and tumor-wise RNA and protein expression. FIG. 2A: Gene-wise RNA and protein expression correlation in APOLLO cohort, 87 tumors and 7,472 co-detected genes. FIG. 2B: Gene-wise RNA and protein correlation comparison between APOLLO and CPTAC cohorts, 106 tumors, over 6,729 common, expressed genes between the cohorts. FIG. 2C: Pathway enrichments according to gene-wise RNA and protein expression correlation in APOLLO cohort. FIG. 2D: Tumor-wise RNA:protein expression correlation in APOLLO and in CPTAC cohorts (n=105 with CPTAC DNA-WGS). Tumors displayed in columns and molecular features as rows. Tumor features tested for association with tumor-wise RNA:protein correlation by Spearman correlation tests for continuous variables and by Kruskal-Wallis tests for categorical variables.

FIG. 3A-3E depict RNA and protein expression determinants of patient survival. FIG. 3A: Comparison of log hazard ratios between RNA expression and protein expression on matched genes in APOLLO cohort. Points outside these axis scale (less than 2 or greater than 2) are plotted as 2. ρ and p refer to Spearman rho correlation and test, respectively. Other refers to genes not associated with survival. FIG. 3B: Overall and metastasis free survival by survival signatures in APOLLO cohort (n=83), high/low refer to 50% percentile split, p refers to Cox proportional hazards Wald test of signature score. Upper panels are signatures based on survival proteins and survival RNAs, lower panels are signatures based on survival RNA-proteins. FIG. 3D: CPTAC cohort survival following same layout as FIG. 3B. FIG. 3C and FIG. 3E: Gene-wise RNA:protein correlation across survival gene sets compared by Kruskal-Wallis tests, p*.

FIG. 4A-4C depicts the molecular subtype characteristics and survival outcomes. FIG. 4A: RNA expression subtypes. Tumors (n=87) appear in columns, clinical and genomic features in rows. Protein refers to MS proteomics. Continuous features analyzed by Kruskal-Wallis tests. Categorical features analyzed by Fisher's Exact tests. RNA and protein expression compared by Spearman correlation tests. RNA refers to RNA-seq expression, protein refers to MS-based protein expression. FIG. 4BA: Proteogenomic expression analysis of RNA expression subtypes. Columns indicate molecular enrichment (RNA, Protein, Phosphoprotein by subtype), rows indicate gene sets. FIG. 4C: Survival outcomes of RNA expression subtypes and histological subtypes, analyzed by log-rank tests.

FIG. 5A-5D depicts the proteogenomic network characterization of subtypes. FIG. 5A: Kinase enrichments based on known kinase-substrate links to measured MS-based proteomics and RPPA phosphoresidues. Triangles indicate significant kinase enrichments in either PI, PP, or TRU subtypes (combined FDR<0.01). FIG. 5B-5D: Regulatory networks for Proximal Inflammatory (PI), Terminal Respiratory Unit (TRU) and Proximal Proliferative (PP) subtypes. Box to right indicates network layout (top) and node/edge shape, size, and color schemes (bottom): node shapes indicate molecule types or pathways; red outlines identify nodes significantly associated with the subtype (gray otherwise); blue-to-red shading indicates node association/enrichment with subtype (gray denotes no measured data); enlarged diamonds and “vee” shapes indicate enriched kinases and mutated genes, respectively; red outlined triangles with italic text labels indicate TFs identified from TF enrichment analysis; and edge color represents types of protein-protein or protein-pathway links.

FIG. 6 depicts the proteogenomic features associated with subtype networks. Individual features associated with LUAD subtypes and networks (related to FIG. 5). Sample-wise somatic alterations in KEAP1, STK11, SMARCA4, TP53, KRAS, and EGFR with black triangles to the right indicating significant enrichment of molecular alterations in the given subtype (Fisher's exact test p<0.05) and black diamonds indicating significantly recurrent somatic mutations in the subtype (MutEnricher FDR<0.1). Additional panels display select individual molecular features associated with the subtypes.

FIG. 7 depicts predictor performance in the APOLLO cohort. Survival scores were calculated based on the relative expression levels of 14 aggressive lung cancer-related molecules provided in Table 2, which were then divided into three risk groups (low, intermediate, or high) representing three tertiles. Survival outcomes were compared between pairs of risk groups by log ranks tests (p-value).

FIG. 8 depicts predictor performance in the CPTAC cohort. Survival scores were calculated based on the relative expression levels of 14 aggressive lung cancer-related molecules provided in Table 2, which were then divided into three risk groups (low, intermediate, or high) representing three tertiles. Survival outcomes were compared between pairs of risk groups by log ranks tests (p-value).

### DETAILED DESCRIPTION

Reference will now be made in detail to various exemplary embodiments, examples of which are illustrated in the accompanying drawings and discussed in the detailed description that follows. It is to be understood that the following detailed description is provided to give the reader a fuller understanding of certain embodiments, features, and details of aspects of the disclosure, and should not be interpreted as limiting the scope of the disclosure.

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

### Definitions

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, 1%, ±0.9%, ±0.8%, ±0.7%, ±0.6%, ±0.5%, ±0.4%, ±0.3%, ±0.2% or ±0.1% from the specified value as such variations are appropriate to perform the disclosed methods and/or to make and use the disclosed compositions. When “about” is present before a series of numbers or a range, it is understood that “about” can modify each of the numbers in the series or range.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “at least” prior to a number or series of numbers (e.g., “at least two”) is understood to include the number adjacent to the term “at least” and all subsequent numbers or integers that could logically be included, as clear from context. When the term “at least” is present before a series of numbers or a range, it is understood that “at least” can modify each of the numbers in the series or range.

The term “diagnosis” or “prognosis” as used herein refers to the use of information (e.g., genetic information or data from other molecular tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

As used herein, the phrase “in need thereof” means that the subject has been identified or suspected as having a need for the particular method or treatment. In some embodiments, the identification can be by any means of diagnosis or observation. In any of the methods and treatments described herein, the subject can be in need thereof. In some embodiments, the subject in need thereof is a human seeking treatment for lung cancer, such as lung adenocarcinoma (LUAD). In some embodiments, the subject in need thereof is a human diagnosed with lung cancer, such as LUAD. In some embodiments, the subject in need thereof is a human undergoing treatment for lung cancer, such as LUAD.

As used herein, the term “in some embodiments,” “in certain embodiments,” “in other embodiments,” “in some other embodiments,” or the like, refers to embodiments of all aspects of the disclosure, unless the context clearly indicates otherwise.

The term “measuring” or “measurement” means assessing the presence, absence, quantity or amount of either a given substance within a clinical or subject-derived sample, including the derivation of qualitative or quantitative concentration levels of such substances, or otherwise evaluating the values or categorization of a subject's clinical parameters. Alternatively, the term “detecting” or “detection” may be used and is understood to cover all measuring or measurement as described herein.

The term “metastasis,” as used herein, refers to the condition of spread of cancer from the organ of origin to additional distal sites in the patient. The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler et al., Adv. Cancer Res., 1978, 28:149-250; Liotta et al., Cancer Treatment Res., 1988, 40:223-238; Nicolson G. L., Biochim. Biophy. Acta, 1988, 948:175-224; Zetter N., Eng. J. Med., 1990, 322:605-612). Increased malignant cell motility has been associated with enhanced metastatic potential in animal as well as human tumors (Hosaka et al., Gan., 1978, 69:273-276; Haemmerlin et al., Int. J. Cancer, 1981, 27:603-610).

As used herein, the term “molecular subtype” refers to a term used to describe the smaller groups that a type of cancer can be divided into, based on whether certain genetic changes or other biomarkers are present. For instance, lung adenocarcinoma can be classified into three molecular subtypes with prognostic implications: the terminal respiratory unit (TRU), proximal-proliferative (PP), and proximal-inflammatory (PI) subtypes.

The term “monitoring” as used herein refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication. In particular, the term “monitoring” can refer to lung cancer (e.g., LUAD) staging, lung cancer (e.g., LUAD) prognosis, assessing extent of lung cancer (e.g., LUAD) progression, or monitoring a therapeutic response.

The term “preventive cancer therapy,” as used herein, refers to a therapy that aims to lower a person's risk of developing cancer. Chemoprevention is one type of preventive cancer therapy that uses substances to stop cancer from developing. Examples of medicines used for chemoprevention include, but are not limited to, tamoxifen (Soltamox®) and raloxifene (Evista®) for breast cancer. Aspirin and other non-steroidal anti-inflammatory drugs (NSAIDs) may also be used to lower the risk of many types of cancer in people with an average risk of cancer. In some embodiments, the preventive cancer therapy is chemoprevention.

As used herein, the term “risk” relates to the probability that an event will occur over a specific time period (e.g., a worsening prognosis of lung cancer) and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula ρ/(l−p) where p is the probability of event and (l−p) is the probability of no event) to no-conversion. Alternative continuous measures which may be assessed in the context of the present disclosure include time to health state (e.g., disease) conversion and therapeutic conversion risk reduction ratios.

A “score” is a value or set of values selected so as to provide a normalized quantitative measure of a variable or characteristic of a subject's condition, and/or to discriminate, differentiate or otherwise characterize a subject's condition. The value(s) comprising the score can be based on, for example, quantitative data resulting in a measured amount of one or more sample constituents obtained from the subject, or from clinical parameters, or from clinical assessments, or any combination thereof. In certain embodiments, the score can be derived from a single constituent, parameter or assessment, while in other embodiments the score is derived from multiple constituents, parameters and/or assessments. The score can be based upon or derived from an interpretation function, such as an interpretation function derived from a particular predictive model using any of various statistical algorithms known in the art. In some embodiments, the score is calculated through an interpretation function or algorithm. In some embodiments, the subject is suspected of having expression of a gene that promotes or contributes to the likelihood of acquiring a disease state or whose expression is correlative to the presence of a disease, disorder, or condition. Calculation of score can be accomplished using known algorithms executable in computer program products within equipment used in sequencing or analyzing samples.

As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, the term “individual” or “patient” is used and is intended to be interchangeable with the term “subject.”

A “therapeutically effective amount” or “effective amount” of a composition is a predetermined amount calculated to achieve the desired effect, i.e., to treat, combat, ameliorate, prevent or improve one or more symptoms of lung cancer, such as LUAD. The activity contemplated by the present disclosure includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue.

As used herein, a “tumor sample” refers to a portion, piece, part, segment, or fraction of a tumor, for example, a tumor which is obtained or removed from a subject (e. g., removed or extracted from a tissue of a subject), preferably a human subject. Tumor samples can be obtained from a subject by means including, but not limited to, venipuncture, excretion, ejaculation, massage, biopsy, needle aspirate, lavage, scraping, surgical incision, or intervention or other means known in the art.

### Aggressive Lung Cancer-Related Molecules

The present disclosure is based, at least in part, on the discovery that the genes provided in Table 1 and Table 2, are differentially regulated (e.g., up-regulated or down-regulated) in lung adenocarcinoma (LUAD). In particular, the disclosure is based on the surprising discovery that the aggregate expression of some or all of the genes provided in Table 1, particularly those provided in Table 2, can discriminate patients with LUAD by overall survival and metastasis-free survival. This unexpected finding makes possible to use those genes as biomarkers to diagnose or prognose LUAD, or lung cancer in general. It also makes it possible to use those genes as biomarkers to monitor the progression of LUAD, or lung cancer in general, to predict the effectiveness of a cancer therapy in a subject having LUAD, or lung cancer in general, or to predict clinical outcomes, such as patient overall survival or metastasis-free survival. Because LUAD is a fairly aggressive form of lung cancer, these genes are named “aggressive lung cancer-related molecules” in the present disclosure.

The aggressive lung cancer-related molecules of the present disclosure were identified through deep proteogenomic profiling of 87 LUAD tumors integrating whole genome sequencing, transcriptome sequencing, proteomics and phosphoproteomics by mass spectrometry, and reverse phase protein arrays. A total number of 66 aggressive lung cancer-related molecules were identified and their corresponding log hazard ratio (“coefficient”) in terms of RNA expression and protein expression are summarized in Table 1.

TABLE 1
Sixty-six Aggressive Lung Cancer-Related Molecules
of the Present Disclosure. “Coefficient for RNA”: log
hazard ratio of RNA expression; “Coefficient for
protein”: log hazard ratio of protein expression.

| Gene | Coefficient | Coefficient | Ensembl Gene |
|---|---|---|---|
| Symbel | for RNA | for protein | Identifier |
| ADCY9 | −0.5327 | −0.9918 | ENSG00000162104.9 |
| ADGRF5 | −0.3428 | −0.5720 | ENSG00000069122.18 |
| AHR | −0.7009 | −0.7714 | ENSG00000106546.13 |
| ARSD | −0.4798 | −0.8889 | ENSG00000006756.15 |
| AVEN | 1.1168 | 1.0863 | ENSG00000169857.7 |
| C16orf89 | −0.1840 | −0.3485 | ENSG00000153446.15 |
| CALU | 0.6254 | 0.2775 | ENSG00000128595.16 |
| CAMK2D | −0.8291 | −0.7193 | ENSG00000145349.16 |
| CD302 | −0.7549 | −0.7291 | ENSG00000241399.6 |
| CLIP1 | 1.2274 | 1.3503 | ENSG00000130779.19 |
| COL11A1 | 0.1932 | 0.1983 | ENSG00000060718.21 |
| CPE | 0.2897 | 0.2295 | ENSG00000109472.13 |
| CYP24A1 | 0.1849 | 0.2793 | ENSG00000019186.9 |
| CYP4B1 | −0.2251 | −0.4527 | ENSG00000142973.13 |
| CYP4X1 | −0.3584 | −0.3613 | ENSG00000186377.7 |
| ECHDC2 | −0.8766 | −0.8391 | ENSG00000121310.16 |
| EGFR | −0.4029 | −0.7220 | ENSG00000146648.17 |
| FAAH | −0.5931 | −0.8775 | ENSG00000117480.15 |
| FAM83A | 0.3225 | 0.4414 | ENSG00000147689.16 |
| FDXR | −0.8178 | −0.9254 | ENSG00000161513.11 |
| FLNC | 0.3707 | 0.3898 | ENSG00000128591.15 |
| FMO5 | −0.2633 | −0.6495 | ENSG00000131781.12 |
| FNDC1 | 0.4065 | 0.4550 | ENSG00000164694.16 |
| FOSL2 | 0.8366 | 0.6170 | ENSG00000075426.11 |
| FURIN | 0.4956 | 0.2889 | ENSG00000140564.11 |
| GAPDH | 0.5556 | 0.4697 | ENSG00000111640.14 |
| GFPT2 | 0.5044 | 0.3541 | ENSG00000131459.12 |
| GPD1L | −0.7587 | −0.6108 | ENSG00000152642.10 |
| KIF26B | 0.5076 | 1.0387 | ENSG00000162849.15 |
| KYNU | 0.2649 | 0.3197 | ENSG00000115919.14 |
| MAMDC2 | −0.3899 | −0.4607 | ENSG00000165072.9 |
| MATR3 | −1.4717 | −1.2483 | ENSG00000015479.18 |
| MCCC1 | −0.9775 | −0.8586 | ENSG00000078070.12 |
| MISP | 0.4261 | 0.4248 | ENSG00000099812.8 |
| MTX3 | −1.0991 | −1.0647 | ENSG00000177034.15 |
| MUC4 | 0.2626 | 0.5288 | ENSG00000145113.21 |
| MUC5B | 0.1751 | 0.1804 | ENSG00000117983.17 |
| MYO1E | 0.6436 | 1.2710 | ENSG00000157483.8 |
| NAMPT | 0.6266 | 0.4829 | ENSG00000105835.11 |
| NDRG2 | −0.7352 | −0.6666 | ENSG00000165795.23 |
| NNMT | 0.5023 | 0.5298 | ENSG00000166741.7 |
| NRP2 | 0.5264 | 0.5551 | ENSG00000118257.16 |
| PDE3A | 0.3873 | 0.3662 | ENSG00000172572.6 |
| PLLP | −0.4218 | −0.5167 | ENSG00000102934.9 |
| POSTN | 0.4697 | 0.3598 | ENSG00000133110.14 |
| PPP2R5A | −0.7897 | −0.9960 | ENSG00000066027.11 |
| PUS1 | 1.2534 | 0.6747 | ENSG00000177192.13 |
| RAB29 | −0.6823 | −0.5571 | ENSG00000117280.12 |
| RHOF | 0.4974 | 0.7186 | ENSG00000139725.7 |
| RPS6KA5 | −1.1922 | −1.3443 | ENSG00000100784.10 |
| SDS | 0.4603 | 0.5213 | ENSG00000135094.10 |
| SERPINE2 | 0.3695 | 0.3616 | ENSG00000135919.12 |
| SHROOM2 | −0.6137 | −0.7731 | ENSG00000146950.12 |
| SLC27A3 | −0.6697 | −0.7993 | ENSG00000143554.13 |
| SLC29A1 | −0.9293 | −0.7085 | ENSG00000112759.17 |
| SLC7A5 | 0.4990 | 0.5425 | ENSG00000103257.8 |
| SRPRA | 1.2833 | 0.9370 | ENSG00000182934.11 |
| ST3GAL4 | 0.7268 | 0.5250 | ENSG00000110080.18 |
| SULF1 | 0.4279 | 0.3982 | ENSG00000137573.13 |
| SUSD2 | −0.3182 | −0.3595 | ENSG00000099994.10 |
| THBS1 | 0.5456 | 0.2633 | ENSG00000137801.10 |
| THBS2 | 0.4616 | 0.3442 | ENSG00000186340.14 |
| TMX4 | −0.8894 | −1.3061 | ENSG00000125827.8 |
| TOR1AIP1 | −1.7153 | −1.0618 | ENSG00000143337.18 |
| UTRN | −1.0307 | −1.1217 | ENSG00000152818.18 |
| VCAN | 0.4868 | 0.4991 | ENSG00000038427.15 |

Information related to each gene provided in Table 1 can be obtained via their respective Ensembl Gene Identifier provided in Table 1 through Ensembl website at ensembl.org/Homo_sapiens/Info/Index.

Based on empirical distribution, top 7 positive genes and top 7 negative genes were selected from the 66 genes provided in Table 1 as “top performers” of aggressive lung cancer-related molecules to generate a shorter gene list to facilitate the applications of these aggressive lung cancer-related molecules. The fourteen “top performers” are summarized in Table 2.

TABLE 2
Fourteen “Top Performers” of Aggressive Lung Cancer-Related Molecules
of the Present Disclosure. “Coefficient for RNA”: log hazard ratio
of RNA expression; “Coefficient for protein”: log hazard ratio of protein expression

| Gene | Coefficient | Coefficient | Ensembl Gene | GenBank | UniProtKB |
|---|---|---|---|---|---|
| Symbol | for RNA | for protein | Identifier | Accession No. | Identifier |
| CLIP1 | 1.2274 | 1.3503 | ENSG00000130779.19 | NM_001247997.2 | P30622.2 |
| AVEN | 1.1168 | 1.0863 | ENSG00000169857.7 | NM_020371.3 | Q9NQS1.1 |
| SRPRA | 1.2833 | 0.9370 | ENSG00000182934.11 | NM_003139.4 | P08240.2 |
| PUS1 | 1.2534 | 0.6747 | ENSG00000177192.13 | NM_025215.6 | Q9Y606.3 |
| MYO1E | 0.6436 | 1.2710 | ENSG00000157483.8 | NM_004998.4 | Q12965.2 |
| KIF26B | 0.5076 | 1.0387 | ENSG00000162849.15 | NM_018012.4 | Q2KJY2.1 |
| FOSL2 | 0.8366 | 0.6170 | ENSG00000075426.11 | NM_005253.4 | P15408.1 |
| MATR3 | −1.4717 | −1.2483 | ENSG00000015479.18 | NM_018834.6 | P43243.2 |
| RPS6KA5 | −1.1922 | −1.3443 | ENSG00000100784.10 | NM_004755.4 | O75582.1 |
| TOR1AIP1 | −1.7153 | −1.0618 | ENSG00000143337.18 | NM_015602.4 | Q5JTV8.2 |
| MTX3 | −1.0991 | −1.0647 | ENSG00000177034.15 | NM_001363818.2 | Q5HYI7.2 |
| UTRN | −1.0307 | −1.1217 | ENSG00000152818.18 | NM_007124.3 | P46939.2 |
| TMX4 | −0.8894 | −1.3061 | ENSG00000125827.8 | NM_021156.4 | Q9H1E5.1 |
| MCCC1 | −0.9775 | −0.8586 | ENSG00000078070.12 | NM_020166.5 | Q96RQ3.3 |

Among these 14 aggressive lung cancer-related molecules, CLIP1, AVEN, SRPRA, PUS1, MYO1E, KIF26B, and FOSL2 are up-regulated in LUAD tumors, while MATR3, RPS6KA5, TOR1AIP1, MTX3, UTRN, TMX4, and MCCC1 are down-regulated in LUAD tumors.

### Methods of Use

Because their expression, either on the transcription (i.e., mRNA) level or the translation (i.e., protein) level, is highly correlated with the presence of lung cancer, such as lung adenocarcinoma (LUAD), the aggressive lung cancer-related molecules of the present disclosure are useful in diagnosis, prognosis, monitoring, and/or treating lung cancer, such as LUAD. Moreover, the inventors found that the relative expression level of each of the aggressive lung cancer-related molecules of the disclosure is also highly correlated, either positively or negatively, with the presence of lung cancer, such as LUAD, as well as the clinical outcome, such as patient overall survival and/or metastasis-free survival, a weighted expression, or “coefficient,” which correspond to their log hazard ratio calculated based on the cohort used to select the aggressive lung cancer-related molecules of the disclosure, is assigned to each aggressive lung cancer-related molecule of the disclosure as provided in Table 1 and Table 2. Using the “coefficient” assigned to each aggressive lung cancer-related molecule, a weighted cumulative expression, or aggregate expression, of a plurality of aggressive lung cancer-related molecules selected from Table 1 or Table 2 can be calculated and used as a basis to diagnose, prognose, monitor, and/or treat lung cancer, such as LUAD.

Accordingly, provided herein is a method of identifying the risk of a worsening prognosis of lung cancer in a subject in need thereof, the method comprising, first, measuring relative expression levels of a plurality of aggressive lung cancer-related molecules selected from Table 1 in a tumor sample from the subject, followed by combining the relative expression levels of the plurality of aggressive lung cancer-related molecules to generate a score representing an aggregate expression of the plurality of aggressive lung cancer-related molecules, and then comparing the score to a reference cohort comprising a first group of subjects previously identified as having a low risk of a worsening prognosis of lung cancer and a second group of subjects previously identified as having a high risk of a worsening prognosis of lung cancer, each group having a range of reference scores associated therewith, wherein, if the score is within the range of reference scores associated with the first group, the subject is at low risk of a worsening prognosis of lung cancer, and wherein, if the score is within the range of reference scores associated with the second group, the subject is at high risk of a worsening prognosis of lung cancer. In some embodiments, the reference cohort further comprises a third group of subjects previously identified as having a medium risk of a worsening prognosis of lung cancer, said third group has a range of reference scores associated therewith, and wherein, if the score is within the range of reference scores associated with the third group, the subject is at medium risk of a worsening prognosis of lung cancer.

Also provided herein is a method of predicting the risk of developing metastasis in a subject having lung cancer, the method comprising measuring relative expression levels of a plurality of aggressive lung cancer-related molecules in a tumor sample from the subject, wherein the plurality of aggressive lung cancer-related molecules is selected from Table 1, followed by combining the relative expression levels of the plurality of aggressive lung cancer-related molecules to generate a score representing an aggregate expression of the plurality of aggressive lung cancer-related molecules, and then comparing the score to a reference cohort comprising a first group of subjects previously identified as having a low risk of developing metastasis and a second group of subjects previously identified as having a high risk of developing metastasis, each group having a range of reference scores associated therewith, wherein, if the score is within the range of reference scores associated with the first group, the subject is at low risk of developing metastasis, and wherein, if the score is within the range of reference scores associated with the second group, the subject is at high risk of developing metastasis. In some embodiments, the reference cohort further comprises a third group of subjects previously identified as having a medium risk of developing metastasis, said third group has a range of reference scores associated therewith, and wherein, if the score is within the range of reference scores associated with the third group, the subject is at medium risk of developing metastasis.

In some embodiments, the plurality of aggressive lung cancer-related molecules used for calculating the score comprises at least about 10, such as about 15, 20, 25, 30, 35, 40, 45, 50, 60, or 66 genes selected from Table 1. In some embodiments, about half of the genes selected have a positive coefficient and the remaining genes have a negative coefficient. In some embodiments, the plurality of aggressive lung cancer-related molecules is a subset of the genes provided in Table 1. In some embodiments, the plurality of aggressive lung cancer-related molecules used for calculating the score are selected from Table 2, which includes CLIP1, AVEN, SRPRA, PUS1, MYO1E, KIF26B, FOSL2, MATR3, RPS6KA5, TOR1AIP1, MTX3, UTRN, TMX4, and MCCC. In some embodiments, the plurality of aggressive lung cancer-related molecules used for calculating the score comprises CLIP1, AVEN, SRPRA, PUS1, MYO1E, KIF26B, FOSL2, MATR3, RPS6KA5, TOR1AIP1, MTX3, UTRN, TMX4, and MCCC1. In some embodiments, the plurality of aggressive lung cancer-related molecules used for calculating the score consists of CLIP1, AVEN, SRPRA, PUS1, MYO1E, KIF26B, FOSL2, MATR3, RPS6KA5, TOR1AIP1, MTX3, UTRN, TMX4, and MCCC1.

The relative expression level of each of the plurality of aggressive lung cancer-related molecules in the tumor sample can be measured on the transcription (i.e., mRNA) level or the translation (i.e., protein) level. In some embodiments, the relative expression level of each of the plurality of aggressive lung cancer-related molecules in the tumor sample is measured on the transcription (i.e., mRNA) level and in such embodiments, the score is calculated using the “coefficient for RNA” provided in Table 1 and Table 2. In some embodiments, the relative expression level of each of the plurality of aggressive lung cancer-related molecules in the tumor sample is measured on the translation (i.e., protein) level and in such embodiments, the score is calculated using the “coefficient for protein” provided in Table 1 and Table 2. In some embodiments, the relative expression level of each of the plurality of aggressive lung cancer-related molecules in the tumor sample is measured on the transcription (i.e., mRNA) level and on the translation (i.e., protein) level and in such embodiments, the score associated with the relative RNA expression level is calculated using the “coefficient for RNA” provided in Table 1 and Table 2 and the score associated with the relative protein expression level is calculated using the “coefficient for protein” provided in Table 1 and Table 2.

The relative expression level is based on the expression ratio of a target gene versus a reference gene. A reference gene can be a housekeeping gene in the tumor sample in some embodiments, or an internal control of the instrument used to measure the expression level in other embodiments.

Relative expression levels can be measured using any techniques known in the art. For instance, using sequence information associated with the Ensembl Gene Identifiers provided in Table 1 or Table 2, or the GenBank Accession Nos. provided in Table 2, primers and/or probes can be generated for detecting and/or measuring RNA expression level of the aggressive lung cancer-related molecules. These primers and/or probes can be used in, for example, hybridization analyses, ribonuclease protection assays, and/or methods that quantitatively amplify specific nucleic acid sequences. As an example, Northern hybridization analysis using probes which specifically recognize one or more of the disclosed aggressive lung cancer-related molecules can be used to determine gene expression. Alternatively, expression level can be measured using amplification-based detection and quantitation methods, such as reverse-transcription based polymerase chain reaction (RT-PCR) and PCR. Transcribed RNA of the aggressive lung cancer-related molecules can also be quantified using, for example, other target amplification methods, such as transcription-mediated amplification (TMA), multiplex strand displacement amplification (SDA), and nucleic acid sequence-based amplification (NASBA), or signal amplification methods (e.g., bDNA), and the like. Ribonuclease protection assays can also be used, using probes that specifically recognize mRNA sequences of one or more aggressive lung cancer-related molecules to determine gene expression. Relative quantification of RNA expression can be determined using any methods known in the art, including, but not limited to, relative standard curve method, comparative Ct method, LinRegPCR method, DART-PCR method, Liu & Saint exponential method, and Sigmoid curve-fitting (SCF) method.

Alternatively, expression levels of one or more aggressive lung cancer-related molecules can determined at the protein level using any method known in the art. “Protein” detection comprises detection of full-length proteins, mature proteins, pre-proteins, polypeptides, isoforms, mutations, variants, post-translationally modified proteins and variants thereof, and can be detected in any suitable manner. Protein expression levels can be determined by, for example, measuring the serum levels of peptides encoded by the aggressive lung cancer-related molecules described herein, or by measuring the enzymatic activities of these aggressive lung cancer-related molecules. Such methods are well-known in the art and include, but are not limited to, immunoassays based on antibodies to proteins encoded by the aggressive lung cancer-related molecules, aptamers or molecular imprints. Alternatively, a suitable method can be selected to determine the activity of proteins encoded by the aggressive lung cancer-related molecules according to the activity of each protein analyzed. For proteins, polypeptides, isoforms, mutations, and variants thereof known to have enzymatic activity, the activities can be determined in vitro using enzyme assays known in the art. Such assays include, without limitation, protease assays, kinase assays, phosphatase assays, reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

Once the relative expression level of each of the plurality of aggressive lung cancer-related molecules in a tumor sample is determined, they are combined to generate a score representing an aggregate expression of the plurality of aggressive lung cancer-related molecules. Such an aggregate expression is a weighted cumulative expression of the plurality of aggressive lung cancer-related molecules calculated based on the coefficient (RNA or protein) assigned to each of the aggressive lung cancer-related molecules as provided in Table 1 or Table 2. In some embodiments, the score is calculated by:

$$Score = {\sum }_{i = 1}^{k} {β}_{i} * {expression}_{i}$$

wherein k is the number of the plurality of aggressive lung cancer-related molecules, β is the coefficient assigned to aggressive lung cancer-related molecule i provided in Table 1 or Table 2, and expression is the expression level in the tumor sample. With continuous development of the methods disclosed herein and/or the potential enlargement of the cohort used, the coefficient associated with each of the aggressive lung cancer-related molecules disclosed herein may slightly change. However, such change should be insignificant, and the relative magnitude should be very similar.

The calculated score is then compared to a reference cohort to determine the subject's risk of a worsening prognosis of lung cancer or risk of developing metastasis, which can then be used to make an informed decision as to whether alternative, or additional, cancer therapy is beneficial. The reference cohort generally comprises a group of subjects known to have lung cancer and are being treated for a period of time, such as the APOLLO cohort described in Example 1. Based on their respective overall survival and/or metastasis-free survival, each subject in the reference cohort has a reference score representing their respective aggregate expression of the same plurality of aggressive lung cancer-related molecules with a lower reference score being associated with higher overall survival and/or metastasis-free survival and a higher reference score being associated with lower overall survival and/or metastasis-free survival. Accordingly, in some embodiments, the reference cohort comprises a first group of subjects previously identified as having low reference scores, thus high overall survival and/or metastasis-free survival or low risk of a worsening prognosis or developing metastasis, and a second group of subjects previously identified as having high reference scores, thus low overall survival and/or metastasis-free survival or high risk of a worsening prognosis or developing metastasis. In some embodiments, the reference cohort further comprises a third group of subjects previously identified as having an intermediate reference score, i.e., between the low and high reference scores, thus medium overall survival and/or metastasis-free survival or medium risk of a worsening prognosis or developing metastasis. If the score calculated from the subject in need thereof is within the range of reference scores associated with the first group, the subject is at low risk of a worsening prognosis of lung cancer or low risk of developing metastasis. Conversely, if score calculated from the subject in need thereof is within the range of reference scores associated with the second group, the subject is at high risk of a worsening prognosis of lung cancer or high risk of developing metastasis. If the score calculated from the subject in need thereof is within the range of reference scores associated with the third group, the subject is at medium risk of a worsening prognosis of lung cancer or medium risk of developing metastasis. For the subject identified as high or medium risk of a worsening prognosis of lung cancer, an alternative, or additional, cancer therapy may be beneficial. Accordingly, in some embodiments, the methods disclosed herein further comprise administering a therapeutically effective amount of a lung cancer therapy to the subject identified as having high or medium risk of a worsening prognosis of lung cancer. For the subject identified as having high or medium risk of developing metastasis, a preventive cancer therapy may be beneficial. Accordingly, in some embodiments, the methods disclosed herein further comprise administering a therapeutically effective amount of a preventive cancer therapy to the subject identified as having high or medium risk of developing metastasis. In some embodiments, the preventive cancer therapy comprises a chemoprevention treatment.

In some embodiments, the lung cancer is lung adenocarcinoma (LUAD). LUAD is generally classified into three molecular subtypes with prognostic implications: the terminal respiratory unit (TRU), proximal-proliferative (PP), and proximal-inflammatory (PI) subtypes. Depending on the molecular subtype, the lung cancer therapy administered to the subject may vary. The inventors surprisingly found that a concentration of immune features, such as interferon-γ signaling, PD-L1 protein expression, and high TMB, are associated with the PI subtype; the TRU subtype harbors epidermal growth factor receptor (EGFR) signaling and kinase activity from kinase activity from protein kinase C epsilon (PRKCE) and ribosomal protein S6 kinase A1 (RPS6KA1); and the PP subtype has pronounced cyclin dependent kinase (CDK) activities and upregulation of proteins involved in glycolysis and glutaminolysis. Thus, determining the molecular subtype of LUAD can be clinically important so that therapeutic interventions can be individualized. Accordingly, in some embodiments, the method disclosed herein further comprises, prior to administering the lung cancer therapy, identifying a molecular subtype of the lung adenocarcinoma in the subject. If the molecular subtype of the lung adenocarcinoma is the PI subtype, immunotherapeutic treatments can be more beneficial; if the molecular subtype of the lung adenocarcinoma is the TRU subtype, inhibitory compounds targeting EGFR signaling and/or kinase activity from PRKCE and/or RPS6KA1 can be more beneficial; and if the molecular subtype of the lung adenocarcinoma is the PP subtype, CDK inhibitors and/or glutaminase inhibitors can be more beneficial.

In some embodiments therefore, the methods of the disclosure further comprise, prior to administering the lung cancer therapy, identifying a molecular subtype of the lung adenocarcinoma in the subject and if the molecular subtype is the PI subtype, administering a therapeutically effective amount of one or more immunotherapeutic treatments to the subject. Exemplary immunotherapeutic treatments include, but are not limited to, Nivolumab (OPDIVO®), pembrolizumab (KEYTRUDA®), cemiplimab (LIBTAYO®), Atezolizumab (TECENTRIQ®), durvalumab (IMFINZI®) and/or Ipilimumab (YERVOY®). In some embodiments, the methods of the disclosure further comprise, prior to administering the lung cancer therapy, identifying a molecular subtype of the lung adenocarcinoma in the subject and if the molecular subtype is the TRU subtype, administering a therapeutically effective amount of one or more inhibitory compounds targeting EGFR signaling and/or kinase activity from PRKCE and/or RPS6KA1 to the subject. Exemplary inhibitory compounds targeting EGFR signaling include, but are not limited to, Cetuximab (ERBITUX®), Erlotinib (TARCEVA®), Gefitinib (IRESSA®) Panitumumab (VECTIBIX®) and Lapatinib (TYKERB®). Exemplary inhibitory compounds targeting kinase activity from PRKCE include, but are not limited to, Darovasertib (IDE196). In some embodiments, the methods of the disclosure further comprise, prior to administering the lung cancer therapy, identifying a molecular subtype of the lung adenocarcinoma in the subject and if the molecular subtype is the PP subtype, administering a therapeutically effective amount of one or more CDK inhibitors and/or glutaminase inhibitors to the subject. Exemplary CDK inhibitors include, but are not limited to, Flavopiridol (Alvocidib), Roscovitine, Dinaciclib, P276-00, AT7519, TG02, Roniciclib, and RGB-286638. Exemplary glutaminase inhibitors include, but are not limited to, CB-839 (Telaglenastat). Other cancer therapies, such as Pertuzumab (OMNITARG®), Trastuzumab (HERCEPTIN®), Everolimus (AFINITOR®), Temsirolimus (TORISEL®), Imatinib mesylate (GLEEVEC®), Sorafenib (NEXAVAR®), Dasatinib (SPRYCEL®), Sunitinib (SUTENT®), Nilotinib (TASIGNA®), Pazopanib (VOTRIENT®), Bevacizumab (AVASTIN®), and Sunitinib (SUTENT®) can also be adminstered, along or in combination with any of the above exemplified cancer therapies to the subject.

It was known in the art that the PI subtype of the LUAD is associated with overexpression of the immune cell markers cluster of differentiation 163 (CD163; Ensembl Gene Identifier: ENSG00000177575; GenBank Accession No. NM_203416.4; UniProtKB Identifier: Q86VB7.2) and vascular cell adhesion protein 1 (VCAM1; Ensembl Gene Identifier: ENSG00000162692; GenBank Accession No. NM_001078.4; UniProtKB Identifier: P19320.1), the TRU subtype is associated with overexpression of surfactant protein C (SFTPC; Ensembl Gene Identifier: ENSG00000168484; GenBank Accession No. NM_001317778.2; UniProtKB Identifier: P11686.2) and thyroid transcription factor 1 (NKX2-1 or TTF1; Ensembl Gene Identifier: ENSG00000136352; GenBank Accession No. NM_001079668.3; UniProtKB Identifier: P43699.1), and the PP subtype is associated with overexpression of thymine DNA glycosylase (TDG; Ensembl Gene Identifier: ENSG00000139372; GenBank Accession No. NM_003211.6; UniProtKB Identifier: Q13569.2) and glutathione peroxidase 2 (GPX2; Ensembl Gene Identifier: ENSG00000176153; GenBank Accession No. NM_002083.4; UniProtKB Identifier: P18283.3). Thus, in some embodiments, the molecular subtype of LUAD is identified in the method disclosed herein by overexpression of CD163 and/or VCAM1, if the subtype is the PI subtype, by overexpression of SFTPC and/or NKX2-1 (or TTF1), if the subtype is the TRU subtype, by overexpression of TDG and/or GPX2, if the subtype is the PP subtype. The expression level of these genes can be determined either on the RNA level or the protein level using any methods known in the art, such as those described herein elsewhere.

In a related aspect, also provided herein is a method of treating lung cancer, the method comprising identifying the risk of a worsening prognosis of lung cancer in a subject in need thereof according to any of the methods disclosed herein and administering a therapeutically effective amount of a lung cancer therapy to the subject identified as having high or medium risk of a worsening prognosis of lung cancer. In some embodiments, the lung cancer therapy administered to the subject is the same lung cancer therapy the subject is receiving but with a different dosage regimen, such as an increase dose of the treatment. In some embodiments, the lung cancer therapy administered to the subject is a different lung cancer therapy the subject is receiving. In some embodiments, the lung cancer is LUAD. In some embodiments, the lung cancer is LUAD and the method further comprises identifying a molecular subtype of the LUAD in the subject, as described herein elsewhere, and administering a therapeutically effective amount of a lung cancer therapy according to the molecular subtype of the LUAD identified in the subject. In some embodiments, the molecular subtype of the LUAD identified in the subject is the PI subtype and the lung cancer therapy comprises administering a therapeutically effective amount of one or more immunotherapeutic treatments to the subject. In some embodiments, the molecular subtype of the LUAD identified in the subject is the TRU subtype and the lung cancer therapy comprises administering a therapeutically effective amount of one or more inhibitory compounds targeting EGFR signaling and/or kinase activity from PRKCE and/or RPS6KA1 to the subject. In some embodiments, the molecular subtype of the LUAD identified in the subject is the PP subtype and the lung cancer therapy comprises administering a therapeutically effective amount of one or more CDK inhibitors and/or glutaminase inhibitor to the subject.

In another related aspect, provided herein is a method of treating lung cancer, the method comprising predicting the risk of developing metastasis in a subject having lung cancer according to any of the methods disclosed herein and administering a therapeutically effective amount of a preventive lung cancer therapy to a subject identified as having high or medium risk of developing metastasis. In some embodiments, the preventive cancer therapy comprises a chemoprevention treatment. In some embodiments, the lung cancer is lung adenocarcinoma.

Also provided herein is a method of monitoring effectiveness of a cancer therapy in a subject having lung cancer, the method comprising measuring relative expression levels of a plurality of aggressive lung cancer-related molecules in a tumor sample from the subject as described herein elsewhere before and after the cancer treatment, wherein the plurality of aggressive lung cancer-related molecules is selected from Table 1, and combining the relative expression levels of the plurality of aggressive lung cancer-related molecules before the cancer treatment to generate a pre-treatment score, as described herein elsewhere, representing an aggregate expression of the plurality of aggressive lung cancer-related molecules before the cancer treatment and combining the relative expression levels of the plurality of aggressive lung cancer-related molecules after the cancer treatment to generate a post-treatment score, as described herein elsewhere, representing an aggregate expression of the plurality of aggressive lung cancer-related molecules after the cancer treatment, wherein a lower post-treatment score as compared to the pre-treatment score indicates that the cancer treatment is effective. In some embodiments, the plurality of aggressive lung cancer-related molecules used for calculating the score comprises at least about 10, such as 15, 20, 25, 30, 35, 40, 45, 50, 60, or 66 genes selected from Table 1. In some embodiments, about half of the genes selected have a positive coefficient and the remaining genes have a negative coefficient. In some embodiments, the plurality of aggressive lung cancer-related molecules is a subset of the genes provided in Table 1. In some embodiments, the plurality of aggressive lung cancer-related molecules used for calculating the score are selected from Table 2, which includes CLIP1, AVEN, SRPRA, PUS1, MYO1E, KIF26B, FOSL2, MATR3, RPS6KA5, TOR1AIP1, MTX3, UTRN, TMX4, and MCCC1. In some embodiments, the plurality of aggressive lung cancer-related molecules used for calculating the score comprises CLIP1, AVEN, SRPRA, PUS1, MYO1E, KIF26B, FOSL2, MATR3, RPS6KA5, TOR1AIP1, MTX3, UTRN, TMX4, and MCCC1. In some embodiments, the plurality of aggressive lung cancer-related molecules used for calculating the score consists of CLIP1, AVEN, SRPRA, PUS1, MYO1E, KIF26B, FOSL2, MATR3, RPS6KA5, TOR1AIP1, MTX3, UTRN, TMX4, and MCCC1. In some embodiments, the relative expression levels of the plurality of aggressive lung cancer-related molecules are measured based on protein expression. In some embodiments, the relative expression levels of the plurality of aggressive lung cancer-related molecules are measured based on RNA expression. In some embodiments, the relative expression levels of the plurality of aggressive lung cancer-related molecules are measured based on protein expression and RNA expression. In some embodiments, the subject has lung adenocarcinoma. In some embodiments, the method further comprises changing cancer treatment, such as administering a therapeutically effective amount of a different cancer treatment, to the subject if the post-treatment score is higher than the pre-treatment score.

In another aspect, provided herein is a method of treating LUAD in a subject in need thereof, the method comprising identifying a molecular subtype of the LUAD in the subject, as described herein elsewhere, and administering a lung cancer therapy to the subject according to the molecular subtype of the LUAD identified, wherein the lung cancer therapy comprises administering a therapeutically effective amount of one or more immunotherapeutic treatments, as described herein elsewhere, if the molecular subtype of is the PI subtype, administering a therapeutically effective amount of one or more inhibitory compounds targeting EGFR signaling and/or PRKCE and/or RPS6KA1, as described herein elsewhere, if the molecular subtype is the TRU subtype, or administering a therapeutically effective amount of one or more CDK inhibitors and/or glutaminase inhibitors, as described herein elsewhere, if the molecular subtype of the lung adenocarcinoma is a proximal-proliferative (PP) subtype. In some embodiments, the PI subtype is identified by overexpression of CD163 and/or VCAM1, the TRU subtype is identified by overexpression of SFTPC and/or NKX2-1 (or TTF1), and the PP subtype is identified by overexpression of TDG and/or GPX2.

### EXAMPLES

The following examples are to be considered illustrative and not limiting on the scope of the disclosure described above.

### Example 1. Proteogenomic Analysis of Lung Adenocarcinoma Reveals Tumor Heterogeneity, Survival Determinants, and Therapeutically Relevant Pathways

To characterize the etiology of lung adenocarcinoma (LUAD) in the United States, we performed deep proteogenomic profiling of 87 tumors integrating whole genome sequencing, transcriptome sequencing, proteomics, and phosphoproteomics by mass spectrometry and reverse phase protein arrays. Somatic genome signature analysis revealed three subtypes, including a transition-high subtype enriched with never-smokers, a transversion-high subtype enriched with current smokers, and a structurally-altered subtype enriched with former smokers, TP53 and genome-wide structural alterations. We discovered that within-tumor correlations of RNA expression and protein expression were associated with tumor purity, grade, immune cell profiles, and expression subtype. We detected and then independently validated RNA and protein expression signatures predicting patient survival. Among co-measured genes, more proteins than RNA transcripts had association with patient survival. Integrative analysis characterized three expression subtypes with divergent mutations, proteomic regulatory networks, and therapeutic vulnerabilities. This proteogenomic characterization provides a new foundation for molecularly-informed medicine in LUAD.

### 1. Introduction

Lung adenocarcinoma (LUAD) is a leading cause of cancer deaths in the U.S. (Lin et al., 2018) despite advances in therapeutics targeting somatically-altered genes and immune checkpoints. A major challenge in diagnosing and treating individuals with LUAD is the vast morphological and molecular heterogeneity within and among tumors (de Sousa and Carvalho, 2018; Grilley-Olson et al., 2013; Skoulidis and Heymach, 2019). Several national and international molecular profiling efforts have cataloged a diversity of somatic DNA alterations in LUAD, including driver gene mutations, copy number alterations and fusion genes (Imielinski et al., 2012; Lee et al., 2019a; The Cancer Genome Atlas Research Network, 2014), as well as molecular subtypes defined by RNA expression (The Cancer Genome Atlas Research Network, 2014; Wilkerson et al., 2012; Yatabe et al., 2005). The RNA expression subtypes of LUAD (terminal respiratory unit (TRU), proximal proliferative (PP), and proximal inflammatory (PI)) have distinct clinical outcomes, therapeutic responses and underlying mutations (The Cancer Genome Atlas Research Network, 2014; Wilkerson et al., 2012). Despite these advances, it remains challenging to predict clinical outcomes for all individuals with LUAD based on clinical or molecular characteristics (Bueno et al., 2020; Ringner and Staaf, 2016). In addition, many LUAD tumors do not possess a molecular alteration currently indicated for targeted therapy (Skoulidis and Heymach, 2019).

Recent analyses employing shotgun mass spectrometry (MS)-based proteomics have elucidated new translational and post-translational layers of tumor biology across several tumor types that were not observable through genomics alone (Zhang et al., 2019). The joint characterization of tumor proteomics with genomics and transcriptomics enables proteogenomic analysis, which may enhance our understanding of the molecular mechanisms that drive tumor phenotypes, identify proteome-specific markers of outcome, and elucidate novel treatment paradigms. Initial proteogenomic studies in LUAD, including the NCI's Clinical Proteomic Tumor Analysis Consortium (CPTAC) have described the broad proteogenomic landscape of LUAD including proteomic changes related to mutated genes and protein signaling networks (Chen et al., 2020; Gillette et al., 2020; Sharpnack et al., 2018; Stewart et al., 2015; Xu et al., 2020). However, these studies have also reported a wide range in correlation of relative abundances of RNA expression to protein expression across genes (correlation values: 0.14, 0.17, 0.28, 0.34, 0.53) (Chen et al., 2020; Gillette et al., 2020; Sharpnack et al., 2018; Stewart et al., 2015; Xu et al., 2020). While perfect correlation is not expected due to differential RNA expression regulation versus protein expression regulation, this large range indicates that uncertainty remains in the relationship of protein and RNA levels in LUAD. Precise understanding of the relationship of RNA expression to protein expression in tumors would advance tumor molecular characterization in LUAD and may improve translational biomarker discovery. To date, proteogenomic studies in LUAD have primarily included tumors from individuals in East Asian countries with high rates of non-smoking and EGFR mutation (Chen et al., 2020; Gillette et al., 2020; Xu et al., 2020), which is an important but incomplete segment of the disease. Additionally, clinical follow up data in the published cohorts has been limited and there are few independently validated proteomic markers of clinical outcome in LUAD (Gasparri et al., 2020), compared to the large number available by RNA expression (Ringner and Staaf, 2016).

It is clinically-important to characterize LUAD molecular etiology so that diagnostics and therapeutic interventions can be individualized. To address this aim, we, through the Applied Proteogenomic OrganizationaL Learning and Outcomes (APOLLO) research network (Fiore et al., 2017; Lee et al., 2019b), performed deep proteogenomic profiling of LUAD from a cohort of individuals in the United States unselected for tobacco use. These data were then comprehensively analyzed to identify LUAD's major proteogenomic alterations and subtypes, possible therapeutic vulnerabilities, and molecular discriminants of outcome.

### 2. Results

i. Tumor Collection and Analysis Strategy

Eighty-seven lung adenocarcinomas (LUAD) were selected and acquired from the Lung Cancer Biospecimen Resource Network (lungbio.sites.virginia.edu/) with individual consent and institutional review board approval. LUAD samples were primary tumors that had been surgically-resected for curative intent between 2012 and 2018. Of these, 81% were stage I or II, and 83% were from patients who smoked (Table 3). Tumor histological subtypes were assigned by expert review of matched FFPE sections, revealing three main histologic subtypes—acinar, papillary, and solid. Tumor tissues were then analyzed by five molecular profiling assays: whole genome sequencing (WGS), RNA sequencing (RNA-seq), mass spectrometry (MS)-based proteomics and phosphoproteomics, and reverse phase protein arrays (RPPA). Matched normal tissues were analyzed by DNA WGS. Our analysis strategy involved systematic interrogation of each platform to identify molecular alterations and subtypes. This was followed by integrated proteogenomic analyses to characterize subtypes, to comparatively analyze RNA and protein expression, and to identify molecular discriminants of patient survival.

TABLE 3
Patient characteristics and molecular data types. (A) Patient
and tumor summary statistics. (B) Proteogenomic profiling platforms.
RNA-seq transcript count refers to protein-coding genes with
minimal RNA expression, at least 2 transcripts per million.
Data repository availability indicated by * for closed access
and \hat{ } for open access. Some platforms have
different subsets available by closed and open access.
A. Patient Characteristics

| | Feature | | Summary |
|---|---|---|---|
| | Year of sample collection, range | | 2012-2018 |
| | Race: | White (%) | 92.0% |
| | | Black (%) | 8.0% |
| Sex, % female | | | 48.3% |
| Age at diagnosis, mean ± SD (years) | | | 66.3 ± 10.2 |
| | Smoking history: | current (%) | 23.0% |
| | | former (%) | 59.8% |
| | | never (%) | 17.2% |
| Pack years, mean ± SD (years) | | | 40.4 ± 25.8 |
| | Stage | I (%) | 52.9% |
| | | II (%) | 27.6% |
| | | III (%) | 17.2% |
| | | IV (%) | 0% |
| | | N/A (%) | 2.3% |
| | Tumor grade | 1 (%) | 0% |
| | | 2 (%) | 39.1% |
| | | 3 (%) | 60.9% |
| | Histological subtype: | acinar (%) | 33.3% |
| | | papillary (%) | 18.4% |
| | | solid (%) | 36.8% |
| | | others (%) | 11.5% |

| | |
|---|---|
| % with distant metastasis | 37.9% |
| % lost to follow up before 3 years | 4.5% |
| % lost to follow up before 5 years | 33% |

B. Proteogenomic profiling

| Data type | Platform |
|---|---|
| Germline whole genome sequencing | Illumina * |
| | 39X mean coverage |
| Tumor whole genome sequencing | Illumina *\hat{ } |
| | 116X mean coverage |
| RNA sequencing | Illumina *\hat{ } |
| | 14,374 transcripts |
| MS proteomics | Orbitrap MS \hat{ } |
| | 7,614 proteins |
| MS phosphoproteomics | Orbitrap MS \hat{ } |
| | 10,093 phosphosites |
| RPPA | Aushon 2470 arrayer \hat{ } |
| | 307 antibodies |

ii. Somatic Genome Signature Subtypes Link Molecular Etiologies with Smoking Histories

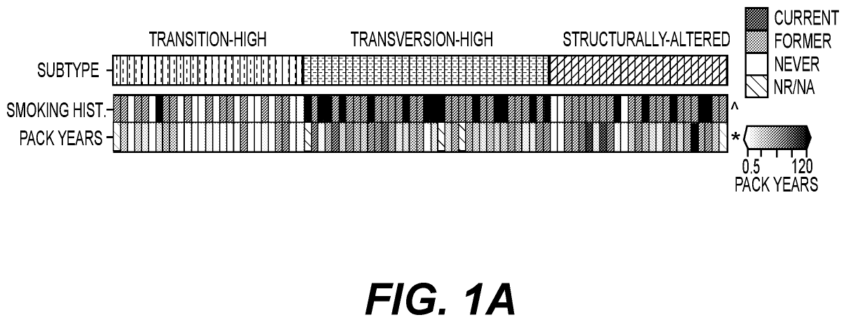

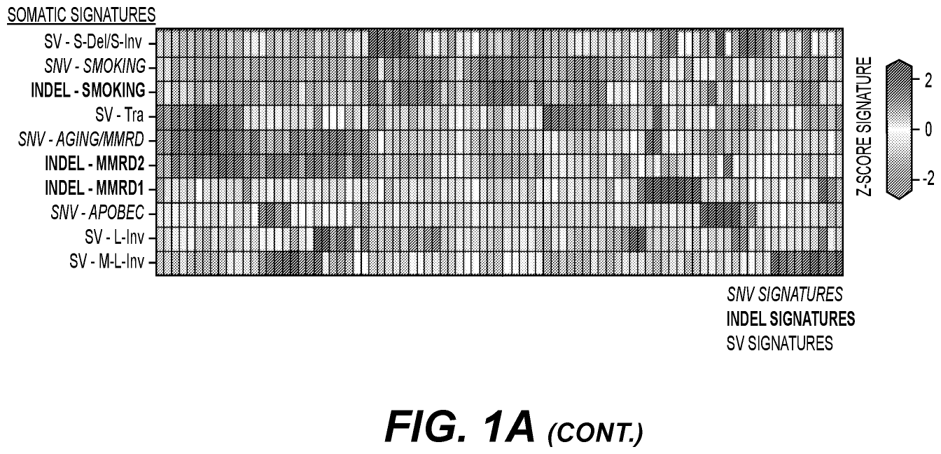

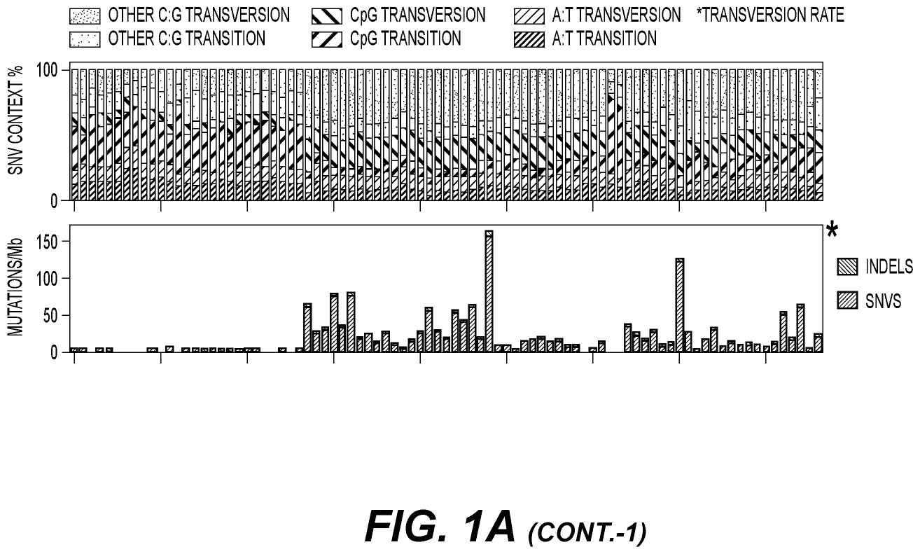

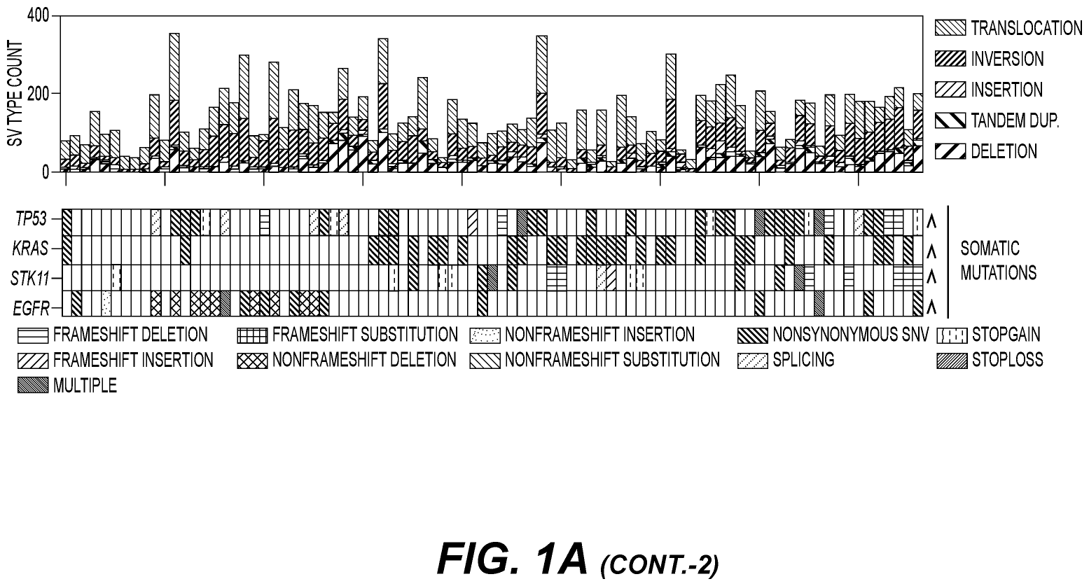

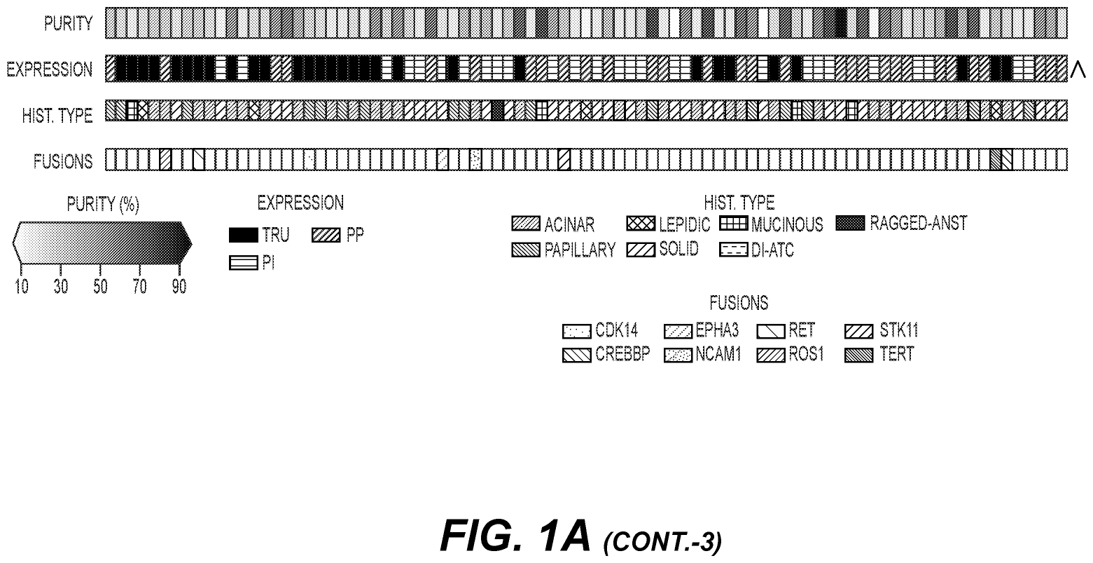

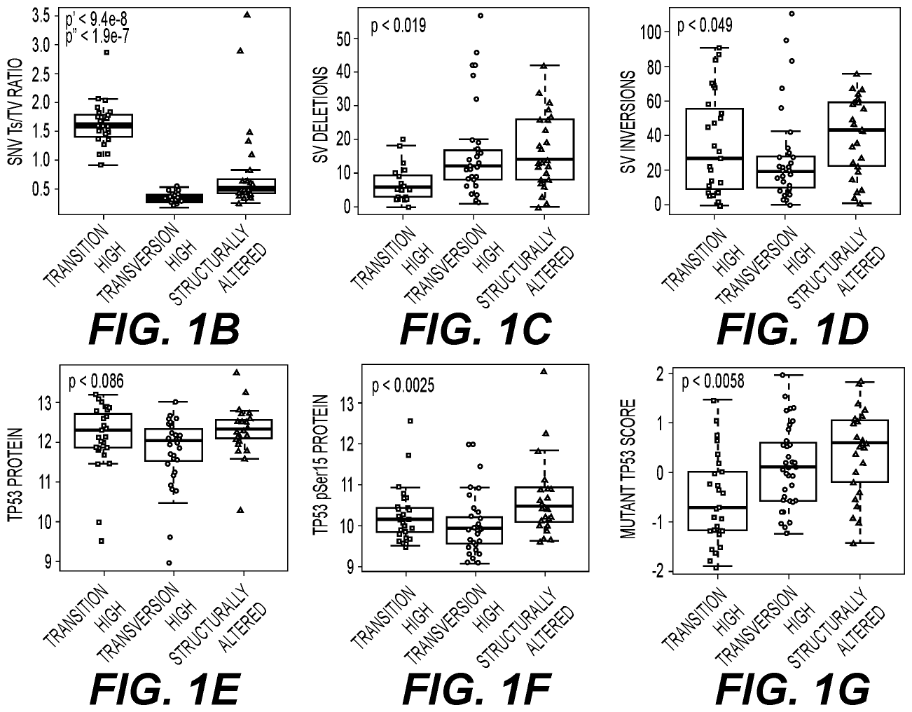

LUAD whole genomes displayed a wide range in tumor mutational burden (TMB) and structural variants (SV) (TMB: 0.35-176 mutations per megabase; SV range: 14-245; FIG. 1A-1G). To identify common patterns among these somatic alterations, we applied a multi-modal correlated topic modeling framework (Funnell et al., 2019) to jointly determine signatures from the frequencies of single nucleotide variant (SNV) base changes in their tri-nucleotide contexts, short insertion and deletion (indel) compositions, sizes, and genomic contexts, as well as SV types and lengths. This analysis revealed three SNV, three indel, and four SV signatures, several of which are associated with known etiologies for specific mutational processes. The three SNV signatures represent established substitution profiles associated with LUAD tumors (Alexandrov et al., 2020; Imielinski et al., 2012): an aging signature characterized by C>T mutations in the NCpG context, a smoking signature comprising C>A transversions, and an APOBEC cytidine deaminase activity signature comprising C>T and C>G mutations in TCN contexts. Among indel signatures, one was similar to the COSMIC signatures ID5 and ID3, the latter of which is associated with tobacco smoking. The other two indel signatures (MMRD1, MMDR2) both resemble DNA replication/repair slippage and have thymine insertions at long homopolymers, with MMRD1 signature also having cytosine and thymine deletions at long homopolymers. The four structural variant signatures were distinguished by long (>10 Mb) inversions, short (1-10 kb) deletions and inversion, medium (100 kb-10 Mb) inversions, and high inter-chromosomal translocation frequencies.

To determine if these somatic genome signatures might identify LUAD subtypes with coordinated mutational processes, we clustered tumors by their signature profiles and identified three signature subtypes (FIG. 1A). We designated these subtypes as “transition-high,” “transversion-high,” and “structurally-altered.” The transition-high subtype was defined by high aging SNV and MMRD2 indel signatures. The transition-high subtype had the greatest SNV transition/transversion ratio (FIG. 1B), the most never-smokers, most tumors with acinar histology, and a very low tumor mutational burden (TMB, median 2.3). The transversion-high subtype was defined by the greatest levels of the smoking SNV and indel signatures and had the greatest enrichment of current smokers and the highest TMB (median 32.7). The structurally-altered subtype was defined by the MMRD1 indel and the medium-long inversion signatures. The structurally-altered signature subtype had the greatest enrichment of former smokers, a high TMB (median 14.7) and intermediate levels of the smoking SNV and indel signatures. Looking further into tumor-wise SV burden, the structurally-altered subtype also had the most structural deletions and structural inversions among these subtypes (FIG. 1C, FIG. 1D). Genomewide somatic copy number alterations resembled published LUAD profiles (2021; The Cancer Genome Atlas Research Network, 2014; Wilkerson et al., 2012) and did not associate with the signature subtypes.

We then interrogated tumor whole genomes for significantly mutated genes, revealing significant enrichments between the signature subtypes. EGFR somatic mutations were enriched in the transition-high subtype (p<0.05), while KRAS and STK11 somatic mutations were enriched in the transversion-high subtype (p<0.05) similar to earlier studies (The Cancer Genome Atlas Research Network, 2014) (FIG. 1A). In contrast, TP53 somatic mutations were most frequent in the structurally-altered subtype (p<0.05), suggesting a causal relationship with this subtype's high structural deletion and inversion events. While TP53 RNA and protein expression were unchanged among the subtypes (FIG. 1E), the structurally-altered subtype, displayed the greatest TP53 pSer15 expression (p<0.0025), a post-translational mark related to DNA damage, consistent with this subtype's high SV burden (Lakin and Jackson, 1999) (FIG. 1F). Concordantly, the structurally-altered subtype exhibited the greatest expression of a mutant TP53 pan-cancer RNA signature (Donehower et al., 2019) (FIG. 1G). These results support that the structurally-altered subtype represents a distinct molecular etiology in LUAD versus being an intermediate between the two other subtypes because it has distinct positively-associated alterations (TP53 mutations, genome inversions, and genome deletions). The pairing of TP53 mutation and structural deletion elevation is consistent with observations from another recent LUAD cohort (Carrot-Zhang et al., 2021a). The structurally-altered subtype described here links those two features with a detailed etiology of mutational signatures, former smoking history, and exclusivity with the transition-high and transversion-high subtypes. In summary, we identified genome signature subtypes that segregated LUAD tumors by coordinated mutational etiologies, never/former/current smoking history, and specific somatically-mutated genes.

Within the non-coding somatic genome, we detected recurrently mutated regulatory regions, some of which were identified as somatic quantitative trait loci with cis genes. Among these was a regulatory element that associated with reduced RNA expression of the surfactants SFTPD, SFTPA1, and SFTPA2. Clustering of somatic structural variant break points identified significantly recurrent events within the STK11 gene locus. These alterations did not associate with the somatic signature subtypes.

iii. Characterization of RNA and Protein Correlations Among Tumors and Across Cohorts

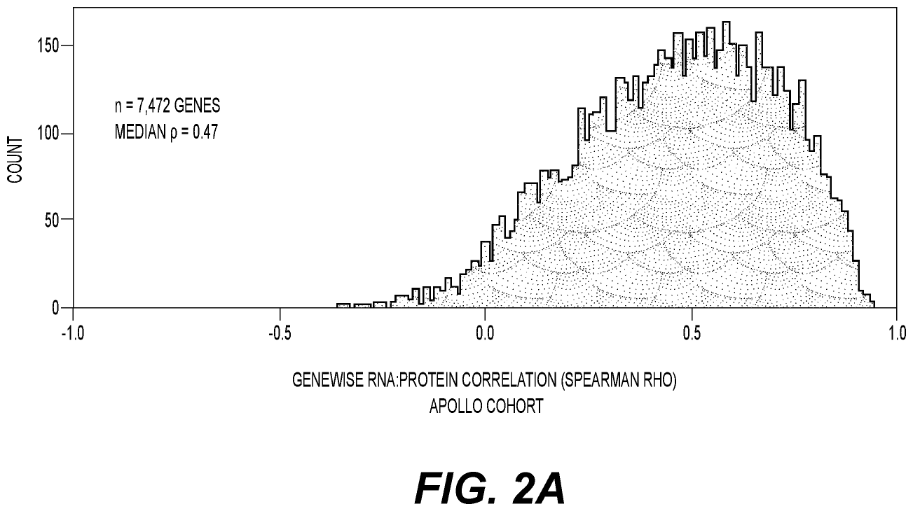

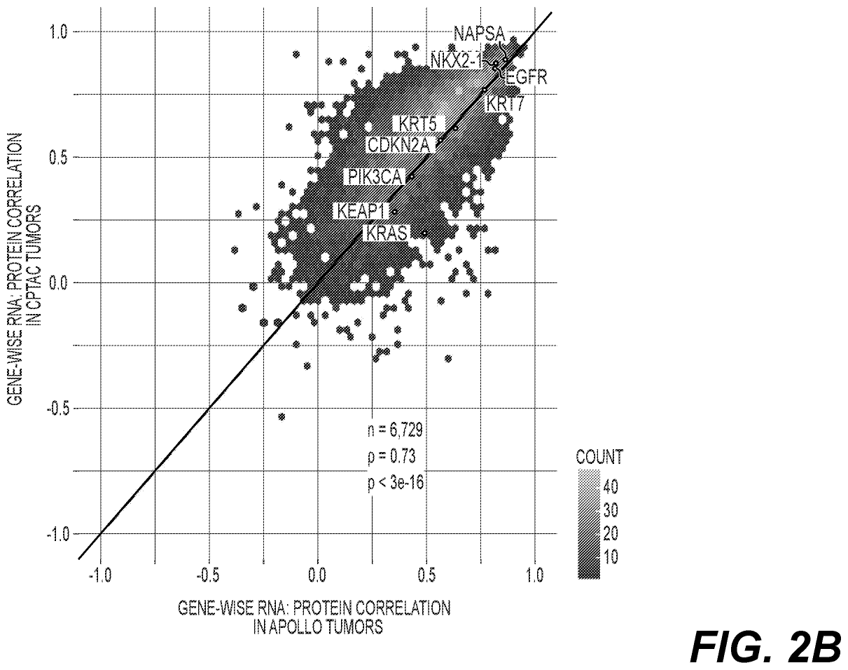

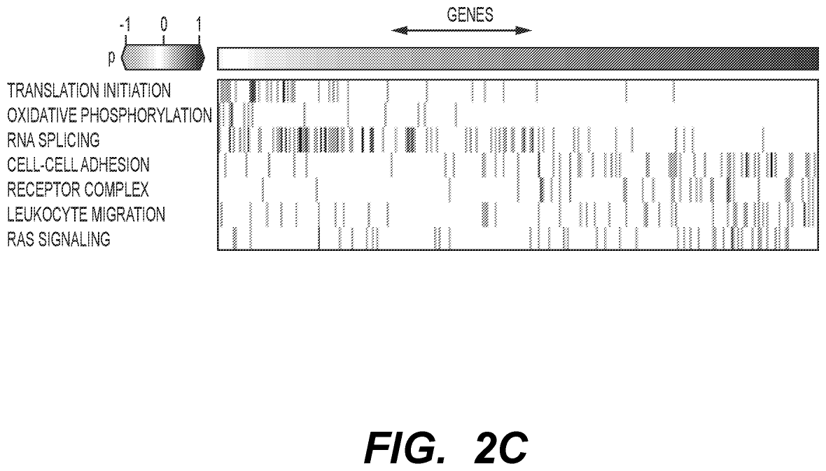

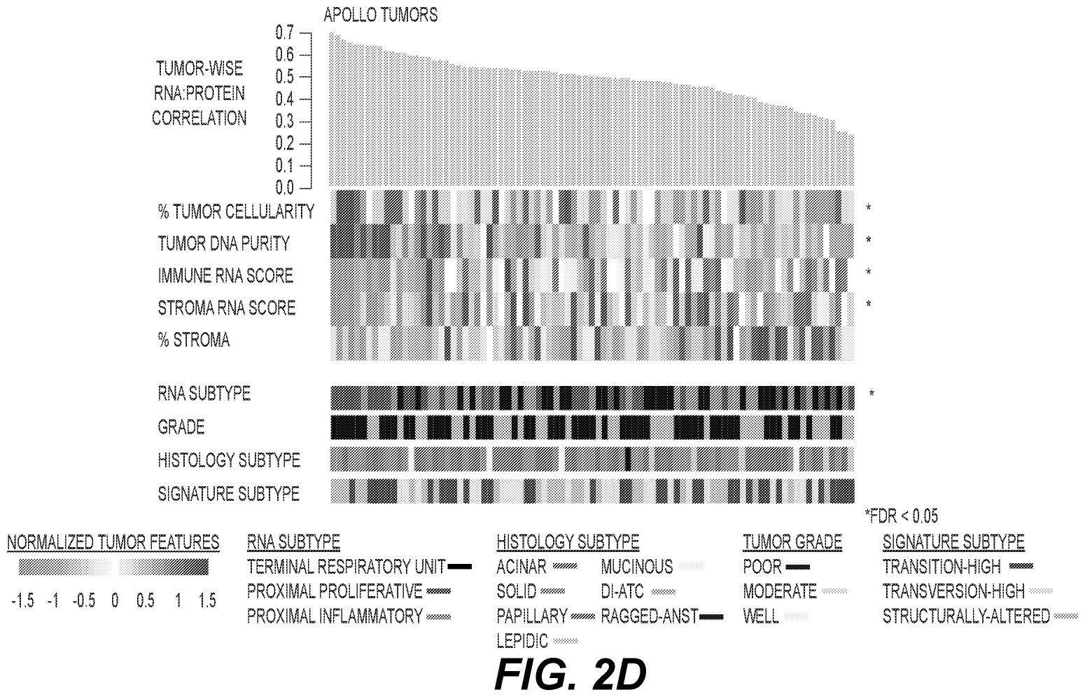

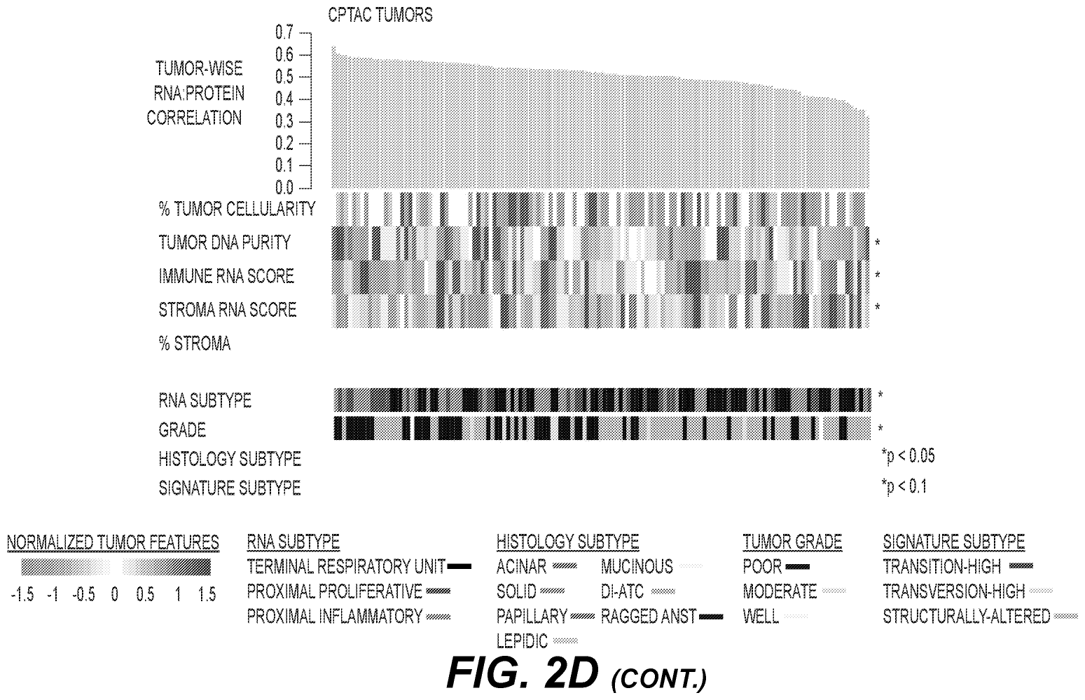

We hypothesized that comparative analysis of protein expression versus RNA expression may reveal differential post-transcriptional regulation across genes and across LUAD tumors. To compare RNA expression and protein expression among all 7,472 co-detected genes, we calculated gene-wise RNA:protein correlations across tumors. The median gene-wise correlation was 0.47 with 84% of genes having statistically significant positive correlation (FIG. 2A). The APOLLO cohort median gene-wise correlation was very similar to the Clinical Proteomic Tumor Analysis Consortium (CPTAC) LUAD cohort's median 0.53 (Gillette et al., 2020), but much larger than other recent studies in LUAD: 0.14 from (Chen et al., 2020), 0.17 from (Sharpnack et al., 2018), 0.28 from (Xu et al., 2020), 0.34 from (Stewart et al., 2015). To determine if gene-wise RNA:protein correlations are conserved across independent LUAD cohorts, we compared our APOLLO cohort to the CPTAC cohort. We found, for the first time, a significant, large positive correlation of gene-wise RNA:protein correlation values between independent LUAD cohorts (ρ=0.73, p<3e-16; FIG. 2B). In addition, these correlations persisted throughout tertile strata of RNA and protein expression, indicating that these distributions are only modestly influenced by absolute abundances (ρ range 0.64-0.78, p<<0.001). Markers of LUAD differentiation, NKX2-1, NAPSA, and KRT7, were among genes with high gene-wise RNA:protein correlation in both cohorts. Different biological pathways were enriched across the range of gene-wise RNA to protein correlations in the APOLLO cohort, similar to other tumor types (Clark et al., 2020; Mun et al., 2019). Highly correlated genes were enriched in cell adhesion and RAS signaling, and poorly correlated genes were enriched in translation initiation and oxidative phosphorylation (FIG. 2C). These enrichments are consistent with observations of tighter control between transcription and translation for genes involved in dynamic cellular processes, which tend to have shorter RNA and protein half-lives, compared to genes involved in housekeeping processes (Schwanhausser et al., 2011; Zhang et al., 2016). Taken together, these data support that transcriptional and translational regulation over many genes is a stable and observable phenomenon across LUAD tumor cohorts and reflective of different biological pathways.

We then sought to identify possible correlation between RNA expression and protein expression across all expressed genes within individual LUAD tumors, called tumor-wise RNA:protein correlations. We found a range of tumor-wise RNA:protein correlations across the APOLLO cohort (ρ range 0.23-0.69), indicating substantial inter-tumor heterogeneity (FIG. 2D). We then compared tumor-wise RNA:protein correlations to molecular properties of cellular heterogeneity and found a positive association with tumor purity estimated from DNA WGS (ρ=0.51, p<8e-7) as well as with tumor cellularity estimates from histological review (ρ=0.37, p<4e-4). In contrast, tumor-wise RNA:protein correlations were negatively correlated with immune and stromal cell RNA expression scores (ρ=−0.54 and ρ=−0.50, respectively; p<2e-6 on each). Tumors with the lowest tumor-wise RNA:protein correlations had elevated percent stroma from histological review in some cases, although not significant (p<0.087). In summary, immune enriched tumors have greater transcriptional and translational variability due to their increased cellular heterogeneity, as compared to tumors with a more uniform population of cancer cells.

To determine if these tumor-wise characteristics are generalizable in LUAD, we analyzed tumor-wise RNA:protein correlations in the CPTAC cohort (FIG. 2D) by the same method, and identified a similar range across tumors (ρ=0.33-0.64) as the APOLLO cohort. Again in the CPTAC cohort, tumor-wise RNA:protein correlation was positively correlated with tumor purity based on WGS somatic mutation signal (ρ=0.26, p<0.0073) and negatively correlated with immune and stroma RNA scores (ρ=−0.255 and ρ=−0.32, respectively, p<0.009 on both). In the CPTAC cohort, we also detected a significant association of poorly differentiated tumors with greater RNA:protein correlations (p<0.002). Therefore, we discover and validate that immune enriched LUAD tumors have greater variability between their RNA and protein levels compared to highly pure tumors.

iv. Transcript and Protein Determinants of Patient Survival

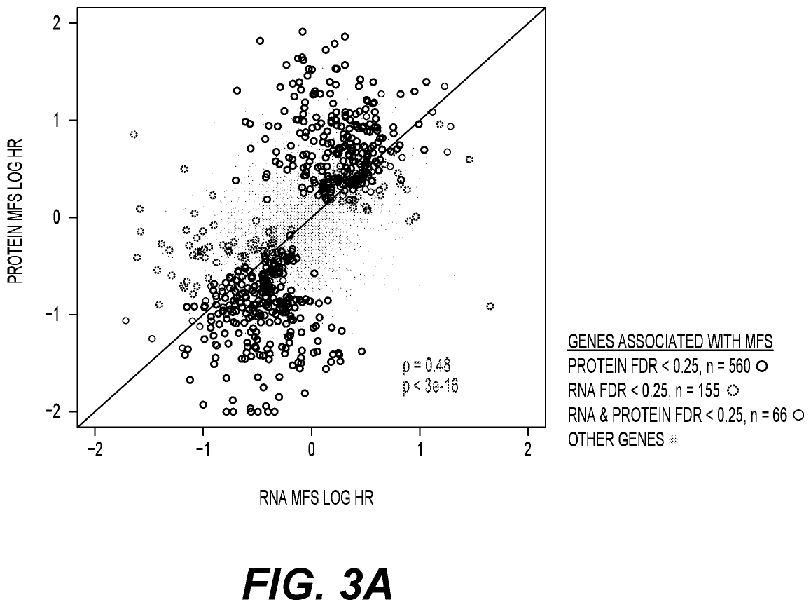

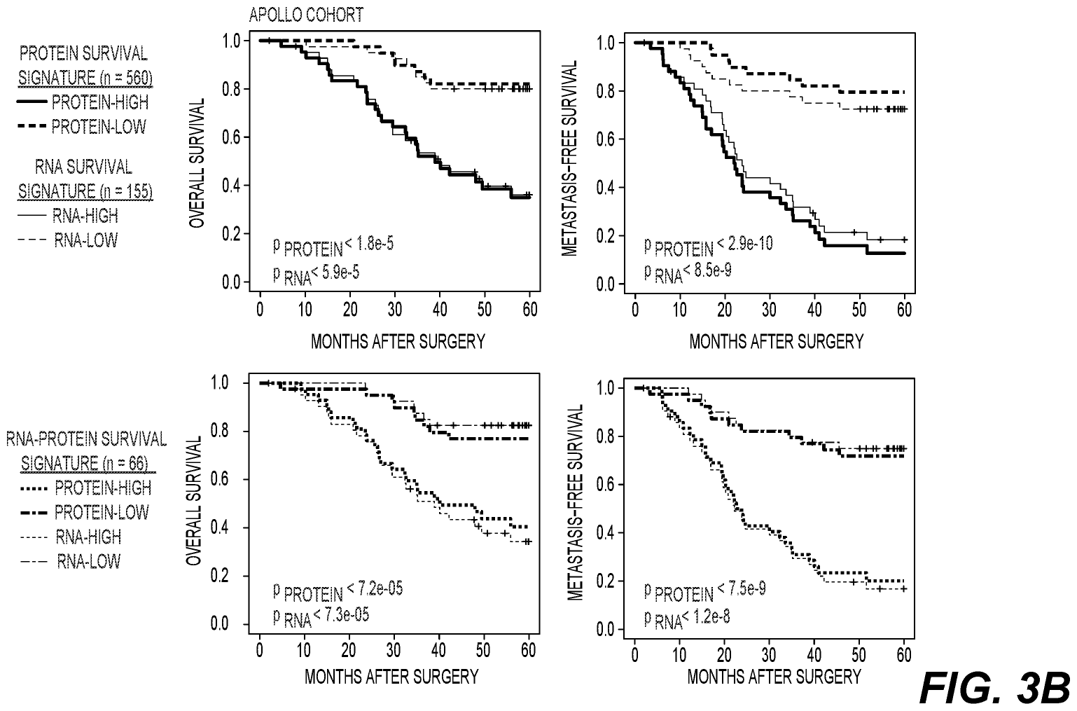

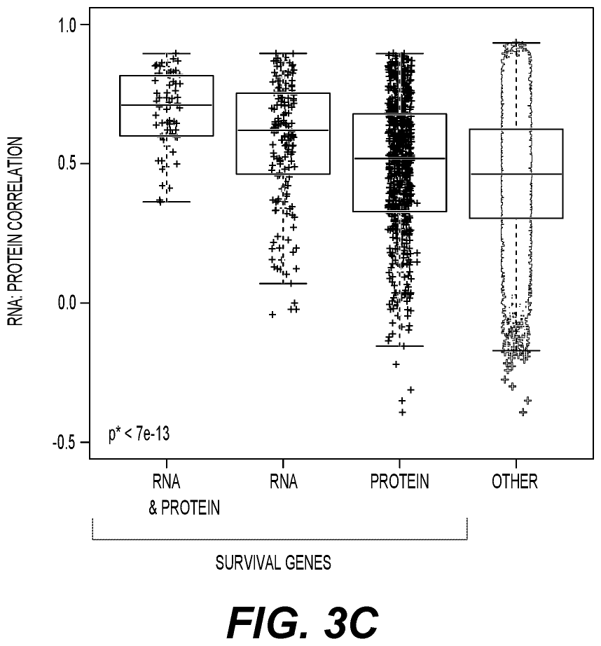

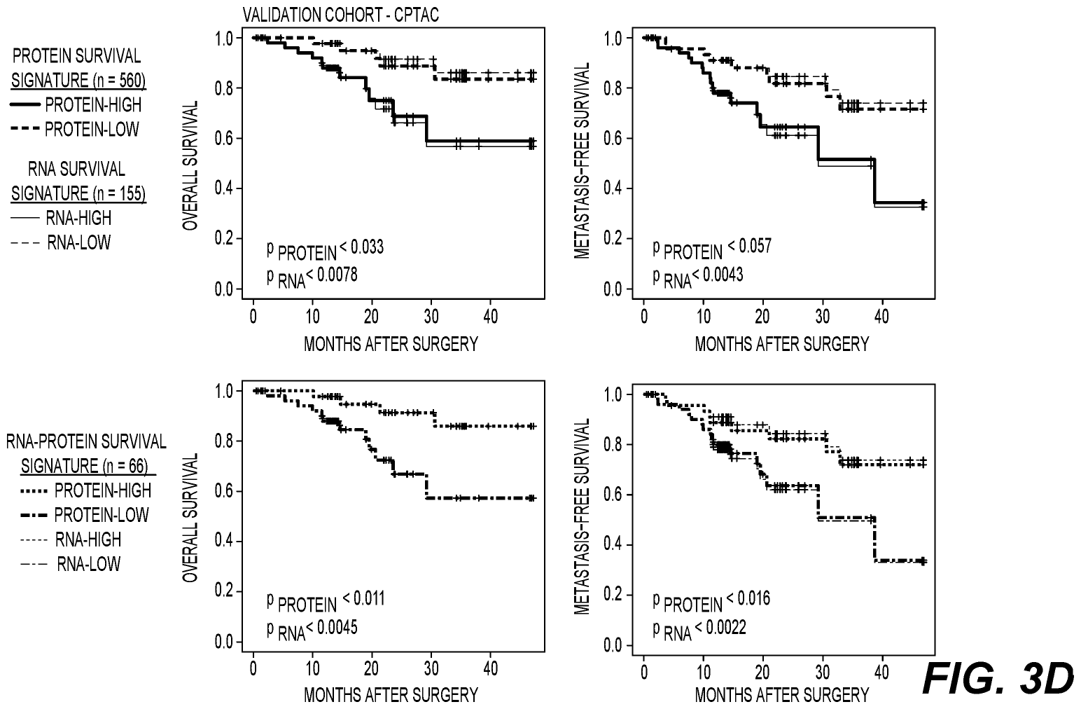

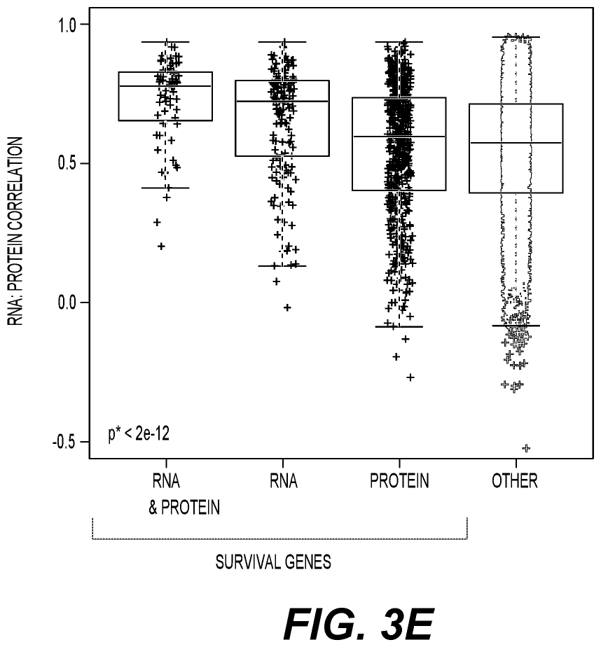

We then sought to identify genes with RNA or protein expression levels that associate with patient survival. Restricting to co-expressed genes (n=7,322), no genes were found that had RNA expression or protein expression significantly associated with patient overall survival (“OS”; FDR<0.25). However, a large number of “survival proteins” were identified as significantly associated with metastasis-free survival (“MFS”; n=560, FDR<0.25). In contrast, fewer “survival RNAs” were associated with MFS (n=155, FDR<0.25). Sixty-six genes were significant by both RNA and protein expression, designated “survival RNA-proteins” and represented a much larger fraction of the survival RNAs than the survival proteins (43% vs 12%). Across all proteins and RNAs, MFS hazard ratios were significantly correlated (FIG. 3B; Spearman ρ=0.48, p<3e-16). This correlation of hazard ratios was larger correlation than a similar analysis performed in prostate cancer (ρ=0.25) (Sinha et al., 2019). Combining survival proteins and their corresponding weights of log hazard-ratios into a protein survival signature and similarly for an RNA survival signature, we found that the aggregate expression of these proteins or these RNAs strongly discriminated patients by OS and MFS (FIG. 3B, OS: protein signature p<1.8e-5, RNA signature p<5.9e-5; MFS: protein signature p<2.9e-10; RNA signature, p<8.5e-9). Additionally restricting to 66 survival RNA-proteins, protein and RNA signatures significantly predicted survival (OS: protein signature, p<7.2e-5; RNA signature, p<7.3e-5; MFS: protein signature, p<7.5e-9; RNA signature, p<1.2e-8). All expression signature remained significantly associated when including tumor stage as a covariate (p<0.05). We then compared gene-wise RNA:protein correlations among survival gene sets and found striking, significant differences (Kruskal-Wallis test, p<7e-13; FIG. 3C). Survival RNA-protein genes had the greatest RNA:protein correlation, followed by survival RNA genes and then survival protein genes. These results support that protein expression has a stronger relationship with patient survival than RNA expression due to a greater number of survival-proteins than survival-RNAs and lower RNA:protein correlation of survival proteins compared to survival RNAs.

To validate these signatures, we then applied the survival signatures to the CPTAC cohort (FIG. 3D). Our survival protein signature was significantly associated with patient OS (p<0.033) and trended significant with patient MFS (p<0.057). The survival protein signature remained significantly associated with OS when including tumor stage as a covariate (p<0.021). The survival RNA signatures significantly associated with patient OS (p<0.0078) and MFS (p<0.0043). Both associations remained significant when including tumor stage as a covariate (p<0.05). Restricting to the survival RNA-proteins, both RNA and protein signatures associated with OS (protein p<0.011, RNA p<0.0045) and MFS (protein p<0.016, RNA p<0.0022), all of which remain significant with tumor stage as a covariate (p<0.05). Validating the APOLLO cohort, survival gene sets had significantly different gene-wise RNA:protein correlation trends in the CPTAC cohort with survival RNA-proteins genes having the greatest RNA:protein correlation followed by survival RNAs, and then survival proteins (Kruskal-Wallis test, p<2e-12; FIG. 3E). Therefore, OS and MFS in patients with LUAD can be predicted by signatures of protein expression and by RNA expression, across independent cohorts.

v. Proteogenomic Subtyping of LUAD

To subtype lung adenocarcinomas by genomewide expression, we first applied the RNA expression subtype predictor (Wilkerson et al., 2012) to assign tumors to the TRU, PI, and PP subtypes (The Cancer Genome Atlas Research Network, 2014; Wilkerson et al., 2012). Applying the predictor to RNA expression or global protein expression resulted in highly similar subtype assignments (p<8e-19). We also performed unsupervised clustering on the cohort's RNA and global protein expression, which also resulted in significantly associated subtype-assignments (p<1.8e-8 in both cases). Furthermore, multi-omic clustering on joint RNA and protein expression also revealed significantly associated subtype-assignments. Therefore, the LUAD tumor subtypes (TRU, PI, and PP) are a robust stratification across both RNA expression and protein expression. To standardize with prior studies (Faruki et al., 2017; Ringner et al., 2016; The Cancer Genome Atlas Research Network, 2014; Wilkerson et al., 2012), we utilized expression subtype assignments based on the RNA subtype predictor applied to RNA-seq throughout the current study.

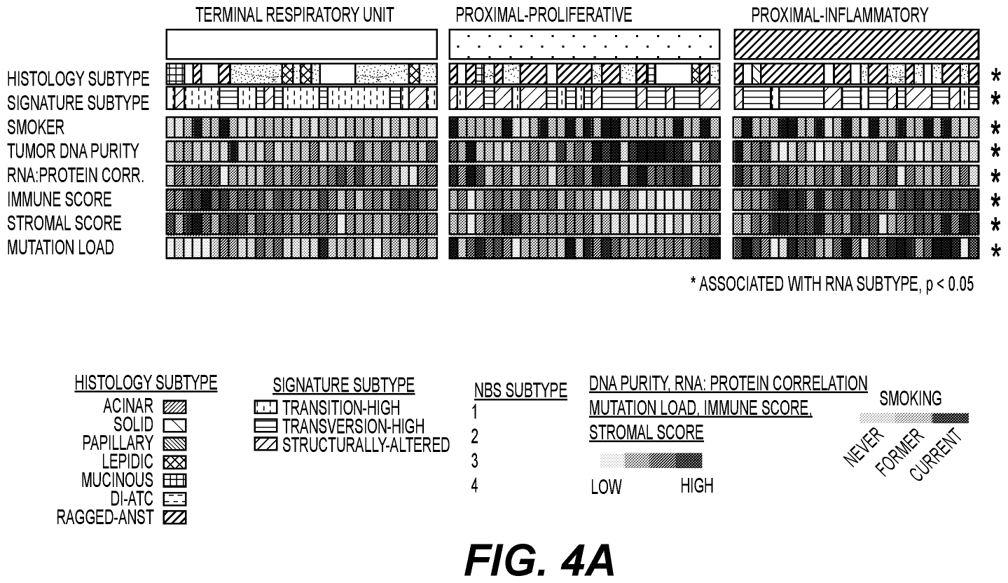

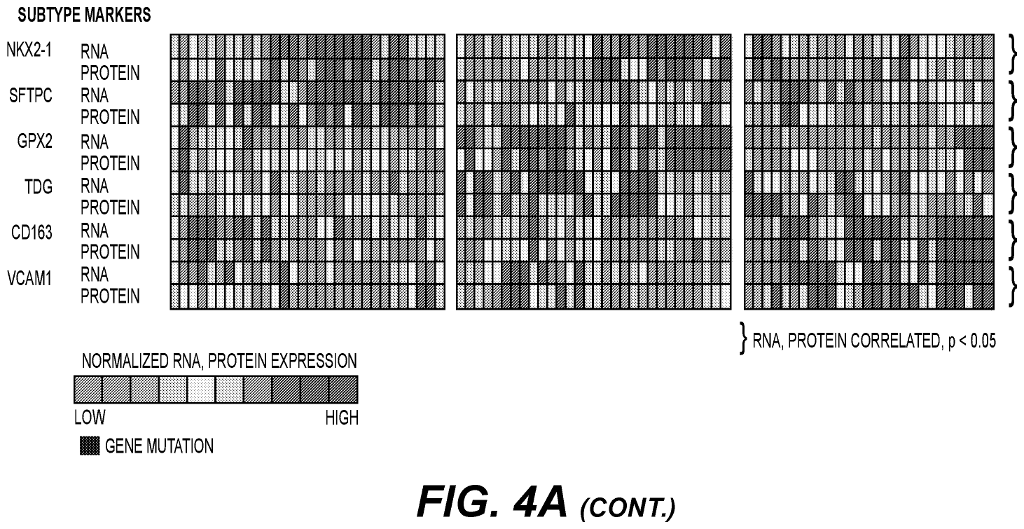

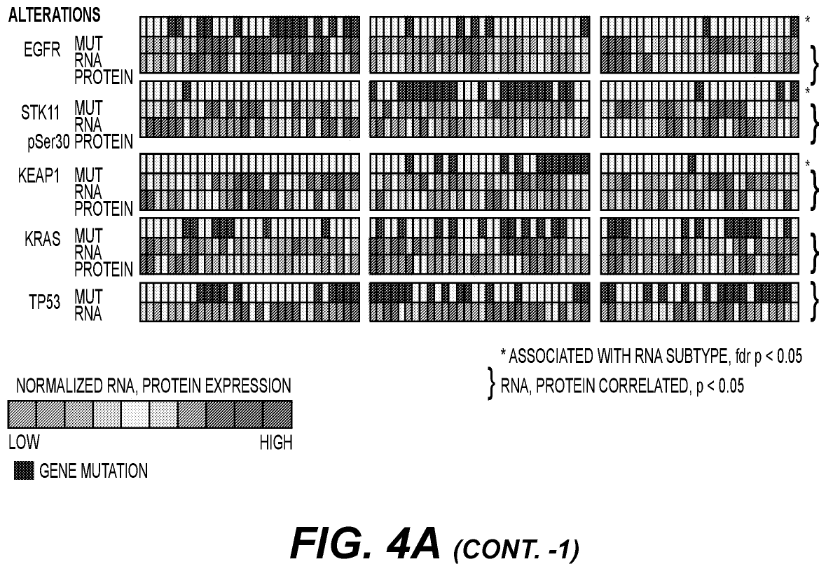

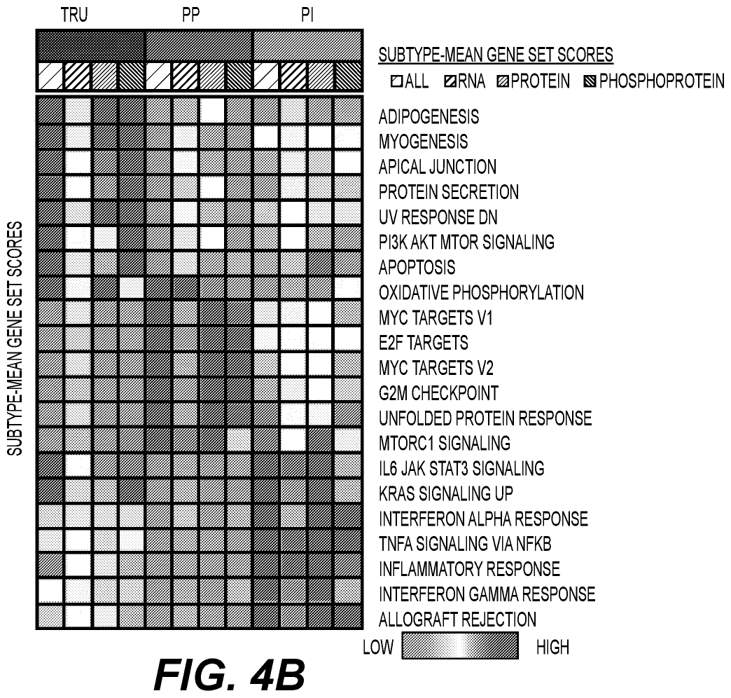

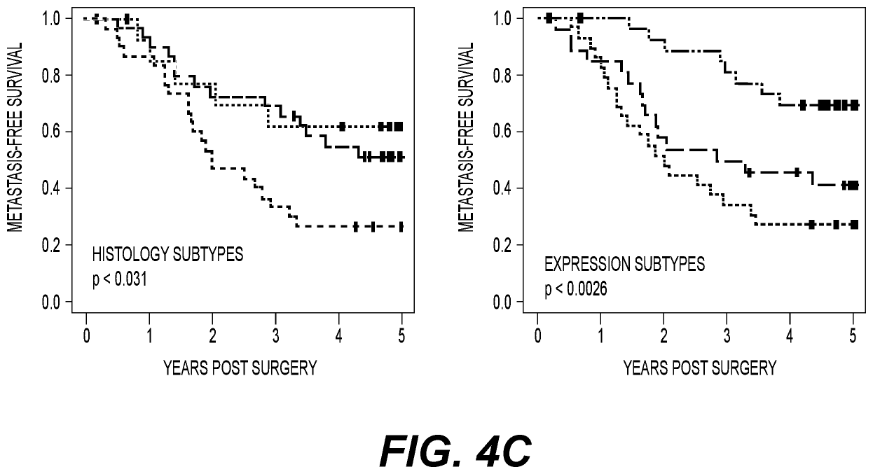

The expression subtypes were enriched with distinct histological subtypes—TRU with acinar and PI with solid, corroborating earlier cohorts (The Cancer Genome Atlas Research Network, 2014; Wilkerson et al., 2012) (FIG. 4A). The expression subtypes overexpressed their canonical, marker genes (Wilkerson et al., 2012) by RNA expression and by protein expression. For example, the TRU subtype overexpressed surfactant protein C (SFTPC) and thyroid transcription factor 1 (NKX2-1 also known as TTF1). The PP subtype overexpressed thymine DNA glycosylase (TDG) and glutathione peroxidase 2 (GPX2). The PI subtype overexpressed the immune cell markers cluster of differentiation 163 (CD163) and vascular cell adhesion protein 1 (VCAM1). Proteogenomic pathway analysis using the synthesis of RNA, protein and phosphoprotein expression data identified distinct over-expressed pathways among the expression subtypes (FIG. 4B). The TRU subtype overexpressed protein secretion and developmental pathways of adipogenesis and myogenesis. The PI overexpressed inflammatory and interferon-γ signaling pathways, in contrast the “immune-cold” PP subtype overexpressed proliferation-related pathways. With few exceptions, the subtype-specific signals were consistent across RNA, protein and phosphoprotein expression, indicating that distinct transcriptional process of the subtypes carry through translation and post-translational regulation.

Three somatically-mutated driver genes were associated with the subtypes (FDR<0.05), STK11 and KEAP1 mutations in PP and EGFR mutations in TRU (FIG. 4A). By transcript and protein expression, EGFR, STK11, and KEAP1, and STK11 (via STK11 pSer30) were also over or underexpressed in their respective subtype (Kruskal-Wallis on enriched subtype vs others, p<0.05). Genome-wide TMB was greatest in PI compared to other subtypes (medians PI: 27.8, PP: 13.3, TRU: 3.2, Kruskal-Wallis p<6e-6) (FIG. 4A). The proportions of high TMB tumors defined by a clinically-used threshold (10 mutations per megabase (Marabelle et al., 2020)) were also significantly different among the subtypes (PI: 78%, PP: 67%, TRU: 23%, Fisher's Exact p-value<5.2e-5). By immune cell scores, the subtypes showed significant differences (p<6e-7) with PI and TRU leading PP, validating an earlier report (Faruki et al., 2017) (FIG. 4A). Tumor purity was greatest in PP (p<0.001). Additionally, we discovered for the first time that the subtypes display different tumor-wise RNA:protein correlations led by PP (PP: 0.52, PI: 0.49, TRU: 0.47, Kruskal-Wallis p<0.05) (FIG. 2A-2D, FIG. 4A). High tumor purity and tumor-wise RNA:protein correlation in PP may be partially explained by reduced immune cell presence in these tumors. In summary, these proteogenomic characteristics and overexpressed pathways revealed new and vastly distinct biology between the subtypes.

Comparing tumor subtypes by patient survival outcomes, we found that the expression subtypes and histological subtypes significantly associated with MFS (FIG. 4C) while somatic genome signature subtypes did not associate with OS or MFS. Therefore, the intrinsic biology captured in LUAD expression subtypes are now a determinant of MFS, expanding on prior reports on OS (Ringner and Staaf, 2016; The Cancer Genome Atlas Research Network, 2014; Wilkerson et al., 2012).

vi. Integrative Network Modeling of LUAD Subtypes

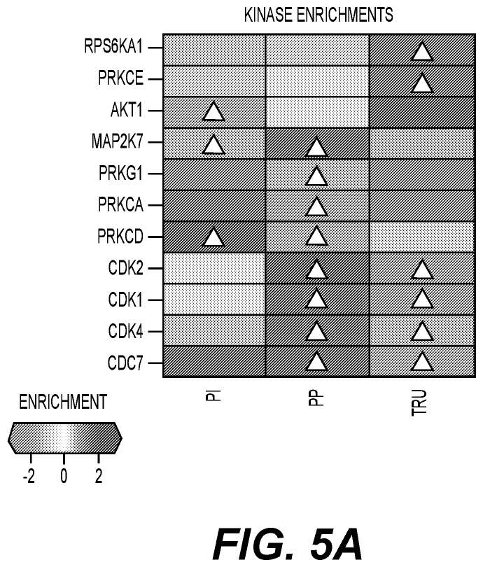

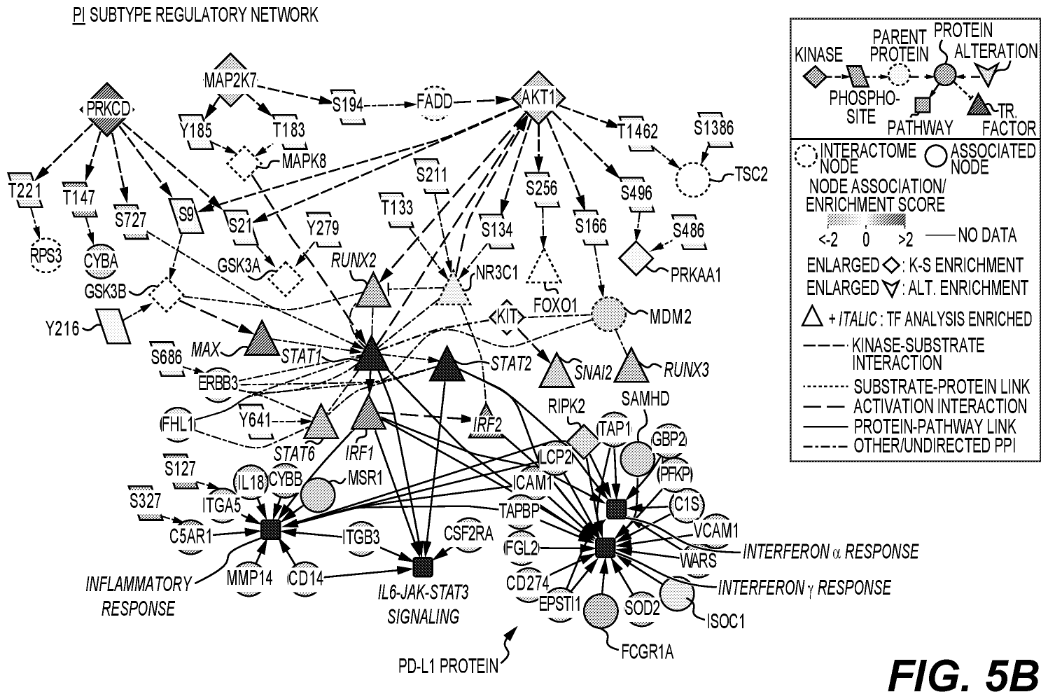

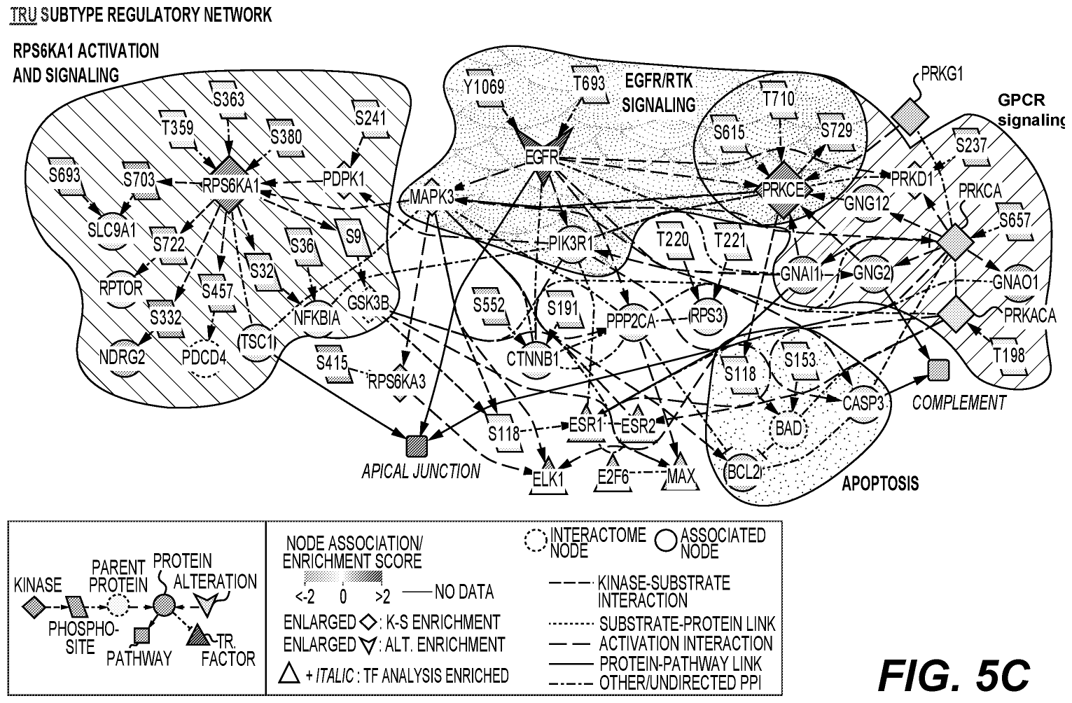

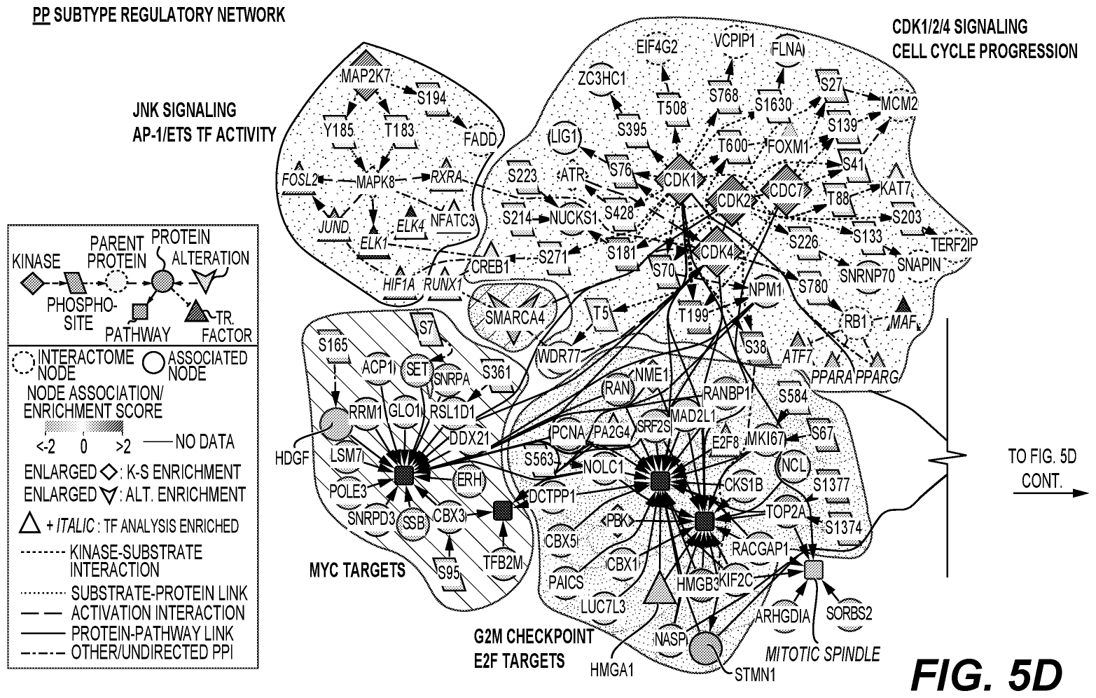

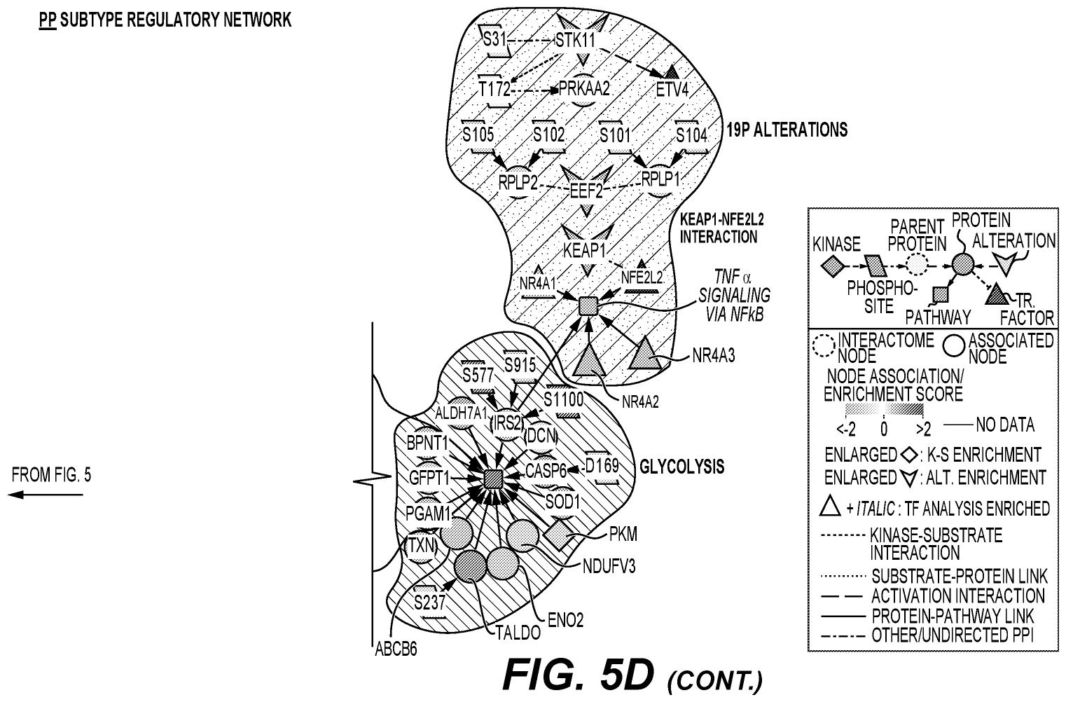

As LUAD tumor expression subtypes are associated with distinct molecular etiologies and patient survival characteristics, we used integrative network modeling to describe subtype-specific proteogenomic signals in the context of known molecular associations with the goal of identifying altered signaling cascades and avenues of potential therapeutic intervention. First, we inferred kinase activities across tumors using known kinase-substrate interactions, which identified subtype-specific enrichments (FIG. 5A). Re-examination of these enrichments with phosphoresidue abundances corrected for global protein expression distinguished direct kinase activity changes from enrichments partially explained by changes in substrate availability. To infer subtype-specific transcription factor (TF) activities, we identified TF motif matches in known LUAD regulatory elements (Corces et al., 2018) and compared these against proximal (−50/+10 kb) gene expression levels corrected for cis copy alterations. These protein kinase and TF enrichments were then integrated with mutated and copy number altered genes, phosphorylation sites, global proteins, and enriched pathways into subtype-specific network models via known kinase-substrate, protein-protein, and protein-pathway interactions.

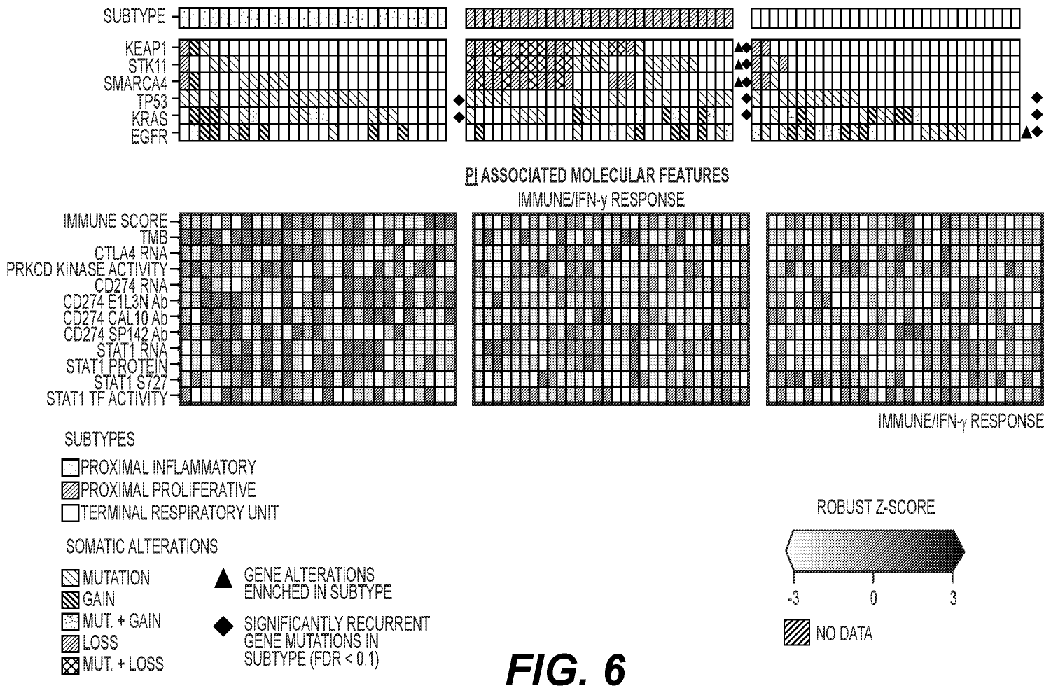

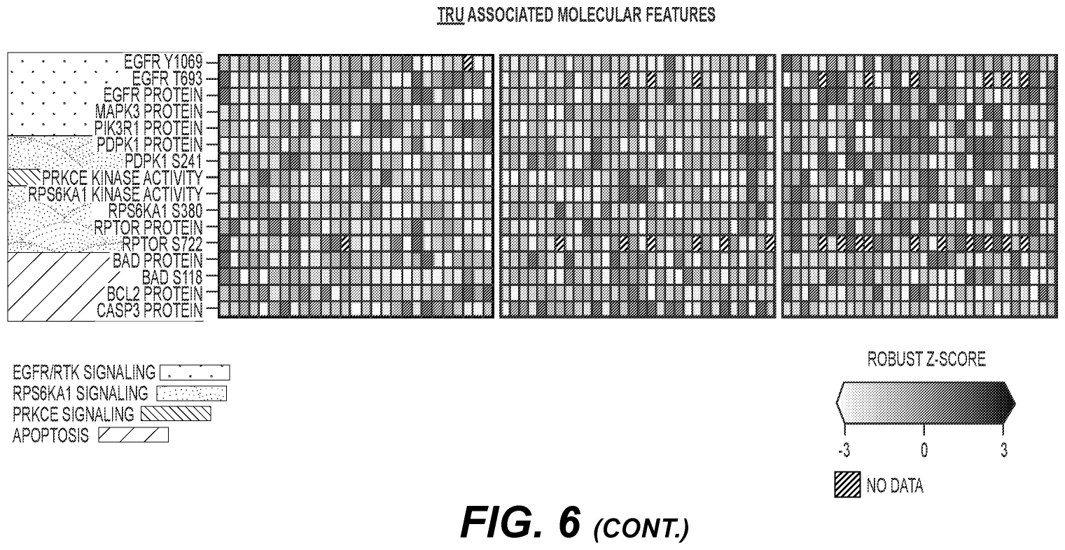

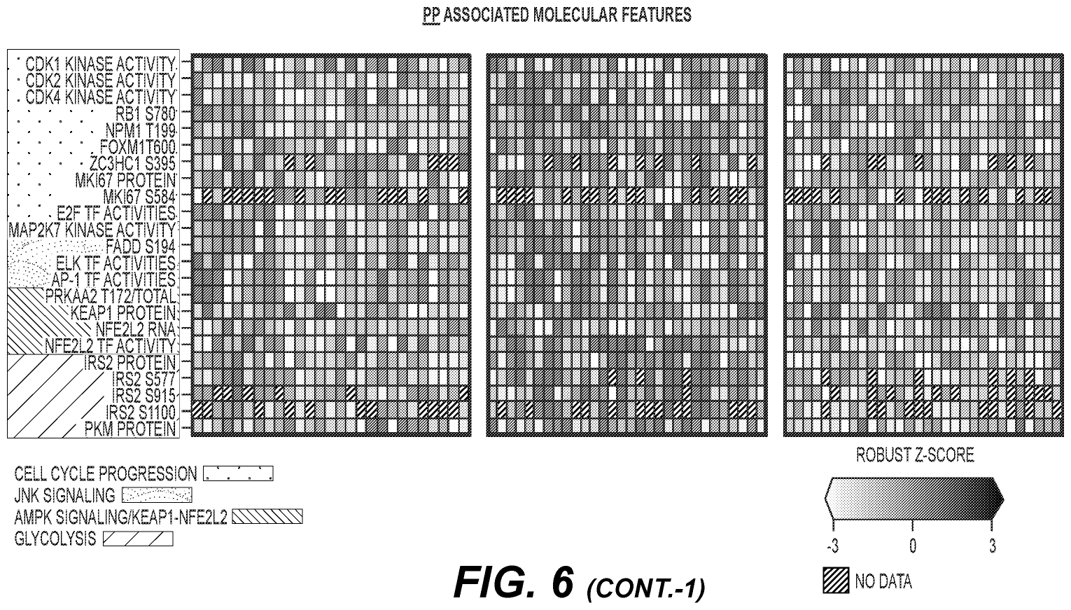

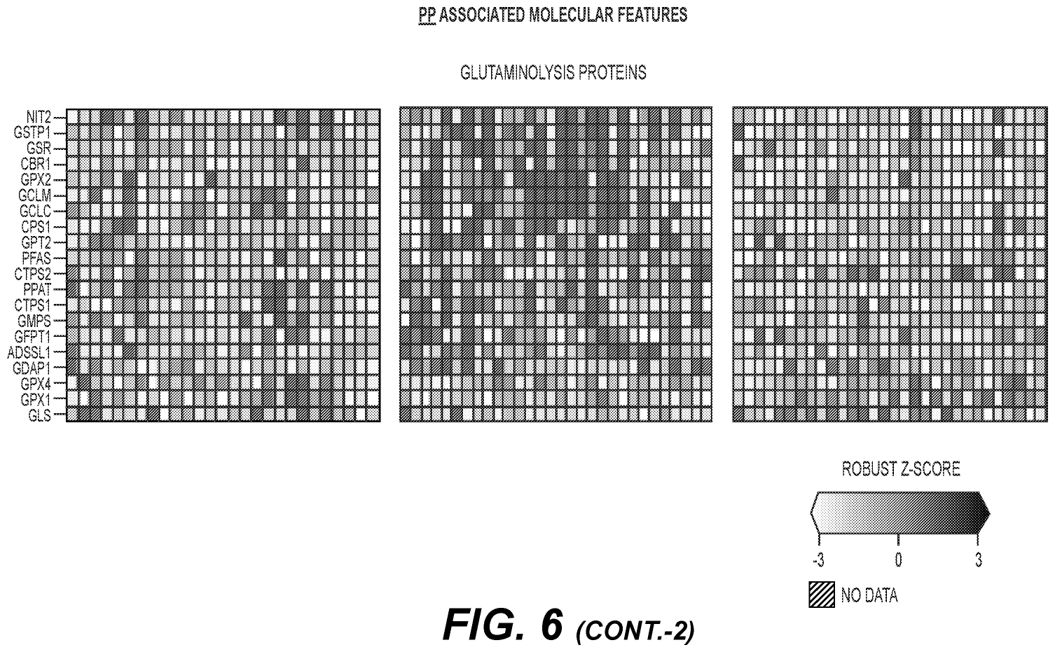

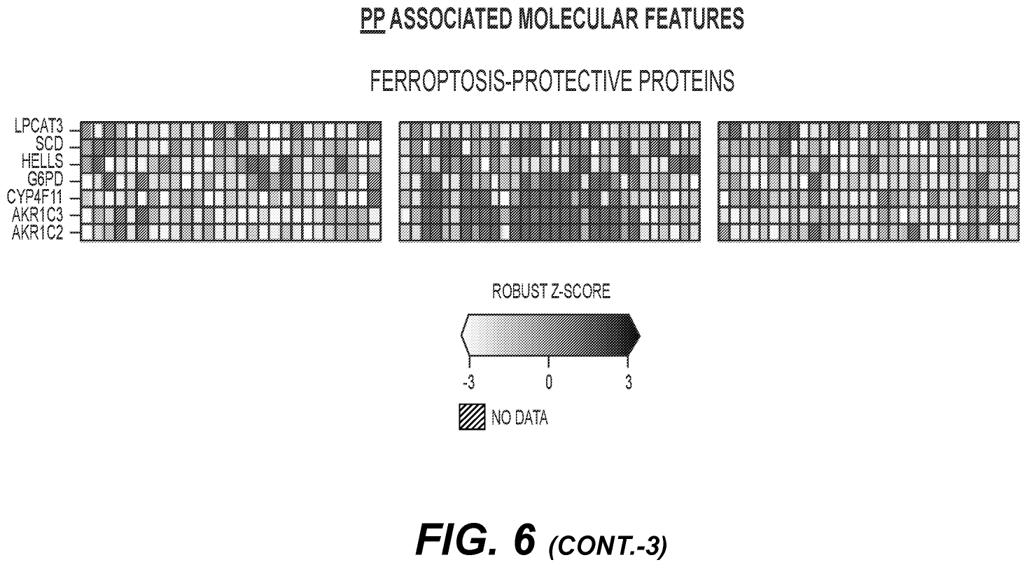

The PI network is characterized by molecular interactions which drive interferon γ (IFN-γ) signaling and inflammation (FIG. 5B). Activated protein kinase C delta (PRKCD) downstream of the IFN-γ receptor phosphorylates a variety of targets in PI, including S727 of STAT1, which is necessary for STAT1's transcriptional activity (Sadzak et al., 2008). Increased STAT1 transcript, global protein expression, and TF activity further supported its activation in PI tumors. STAT1 drives both immunosurveillance, consistent with observed increases in HLA protein and CIITA RNA expression, and immunosuppression, indicated by enhanced RNA expression of the immune inhibitory receptor CD274 (PD-L1) (FIG. 6). The PI subtype also significantly overexpresses the PD-L1 protein, detected using two widely used research based anti-PDL1 antibody clones (E1L3N and CAL10) and the SP-142 clone which is used as an FDA approved companion diagnostic for atezolizumab (FIG. 6). All three anti-PDL1 antibodies have undergone extensive validation testing for clinical tissue-based analysis of PD-L1 levels (Karnik et al., 2018; Parra et al., 2018). Increases in interferon regulatory factor (IRF) TF activities were additionally supported by enhanced IFN-γ and inflammatory signaling in PI. CTLA4 transcript expression, which predominantly derives from CD4+/CD8+ T cells and regulatory T cells in lung tumors (Gentles et al., 2020), was also increased in PI tumors (FIG. 6). Given elevated immune infiltration, high TMB, and enhanced IFN-γ signaling coupled with increased PD-L1 protein and CTLA4 RNA expression, the PI subtype may encapsulate the subset of tumors most likely to respond to immune checkpoint inhibitors.

TRU tumors are characterized by activation of EGFR (FIG. 2A-2D, FIG. 5A-5D). Our network captures activation of ERK (MAPK3 or ERK1) and PI3K-PDK1 (PIK3R1 or p85 and PDPK1) by EGFR signaling as well as downstream activation of RPS6KA1 (aka RSK1 or P90RSK1), which promotes cell proliferation and inhibition of apoptosis (Anjum and Blenis, 2008; Poomakkoth et al., 2016). Enhanced RPS6KA1 kinase activity in TRU tumors is supported in our data by increased phosphorylation of its own residues, including S380, and by several substrate sites, including GSK3B S9, RPTOR S722, NFKBIA (IκBα) S32, and PDCD4 S457. PRKCE activity is also enhanced in TRU. PRKCE has been classified as an oncoprotein due to its antiapoptotic cellular functions (Basu and Sivaprasad, 2007), including inhibitory S118 phosphorylation of pro-apoptotic BAD (FIG. 5C, FIG. 6), which is elevated in TRU. Additionally, increased global protein expression and phosphorylation of several G-protein coupled receptor (GPCR) molecules, PKA (PRKACA global and T198), and PKCα (PRKCA global and S657) were associated with this subtype. Taken together, the TRU subtype captures tumors with marked growth factor signaling that may be most responsive to EGFR inhibitors (Liu et al., 2017) as well as to compounds directed at other members of these signaling cascades (e.g., PRKCE (Astsaturov et al., 2010) or RPS6KA1 (Poomakkoth et al., 2016)).

The network of the PP subtype is characterized by enhanced cell cycle and glycolytic biological processes in an immune-cold microenvironment (Lizotte et al., 2016) (FIG. 2A-2D, FIG. 4A-4C, FIG. 5D, FIG. 6). Pronounced cyclin dependent kinase activities (CDKs 1, 2, and 4 and CDC7) were implicated by enhanced phosphorylation of several target residues, including S780 on RB1 by CDK4 which disrupts inhibition of E2F transcription factors that drive cell cycle progression (Macdonald and Dick, 2012) (FIG. 6). Several of these regulatory states were not implicated by global abundance changes of their respective parent proteins. MAP2K7 kinase activity was also significantly enhanced in PP, reflected by increased phosphorylation at T183/Y185 of MAPK8 and S194 of FADD, the latter of which is associated with G2/M cell cycle regulation (Scaffidi et al., 2000) and poor prognosis in LUAD (Chen et al., 2005). Additionally, global protein and phosphoprotein expression of several proliferation markers were increased in PP, including TOP2A, MKI67, IRS2, and HDGF. Somatic alterations in STK11 and/or KEAP1 encompass 26 of 30 PP tumors, while EEF2 and SMARCA4 alterations are also significantly associated with PP. Inactivation of SMARCA4 can be synthetic lethal with CDK4 (Xue et al., 2019); thus, SMARCA4-altered PP tumors with high CDK4 activity may be responsive to CDK4/6 inhibitor therapies. Metabolic reprogramming was also indicated in PP by upregulation of proteins involved in glycolysis and glutaminolysis, which is consistent with cellular responses to STK11 loss mediated by HIF-1α in conjunction with enhanced cellular stress and reactive oxygen species (ROS) (Faubert et al., 2014). Indeed, coincident STK11-KEAP1 alterations were frequently observed in PP tumors (FIG. 6) as KEAP1 inactivation promotes NFE2L2 activity and antioxidant gene expression (Taguchi and Yamamoto, 2017). While the majority of PP tumors do not possess targetable oncogene mutations, recent studies have demonstrated therapeutic vulnerabilities aimed at PP metabolism, including glutaminase inhibition in STK11-KEAP1-KRAS mutants (Galan-Cobo et al., 2019) and stearoyl-CoA desaturase (SCD) inhibition, which is upregulated in PP tumors along with other ferroptosis-protective molecules (FIG. 6), in combination with ferroptosis inducers in STK11-KEAP1 co-mutants regardless of KRAS status (Wohlhieter et al., 2020).

### 3. Discussion

Here we report the first, large proteogenomic characterization of LUAD from the United States population unselected for tobacco use. Using six molecular profiling technologies, we measured four layers of LUAD biology: genome, transcriptome, proteome, and phosphoproteome. We systematically analyzed these proteogenomic data to identify major alterations, tumor subtypes, signaling patterns, and markers of survival.

By somatic genome signature analysis, we identified three subtypes with coordinated molecular etiologies and tobacco use. The transition-high and transversion-high signature subtypes represent never and current smokers and correspond to tumor groups in earlier cohorts (Alexandrov et al., 2020; Imielinski et al., 2012; Lee et al., 2019a; The Cancer Genome Atlas Research Network, 2014). However, our structurally-altered subtype reveals a novel bifurcation of smokers (former versus current) by a distinct pathway of LUAD mutagenesis, structural genome disorganization and TP53 alterations. An explanation for these observations is that tobacco mutagens produced a moderate transversion signature and a TP53 mutation, individuals quit smoking, and over time structural alterations accumulated due to inhibition of DNA repair checkpoints by the p53 null phenotype to produce tumors despite the lack of continued direct DNA damage by tobacco mutagens. In transversion-high tumors, the continued smoke exposure may have produced a stronger transversion signature and additional sequence mutations such as in KRAS which then led to the development of the tumors. Future studies involving serial sampling of tumors over time would be needed to evaluate this or alternative models. The structurally-altered subtype may have future therapeutic relevance, such as TP53 directed T-cell based therapy (Hsiue et al., 2021).

Our comparative expression analysis of RNA and protein discovered two new characteristics of LUAD. First, we found that gene-wise RNA:protein expression correlation is highly consistent across two independent LUAD cohorts, suggesting broad conservation in transcription rates versus translation rates. These RNA:protein correlations are a resource for biomarker development in selecting an RNA or protein marker for a given gene and for research on expression regulation. Second, we discovered and validated that tumor-wise correlation of RNA and protein expression varies according to immune cellular heterogeneity in LUAD. LUADs with high tumor-wise RNA:protein correlation have high grade similar to clear cell renal carcinoma (Clark et al., 2020), have high purity, and low immune cell content and tend to be in the proximal proliferative subtype. In contrast, LUADs with low tumor-wise RNA:protein correlation have low grade, low purity and high immune cell content. Increasing immune cellular heterogeneity in certain LUADs may present a greater diversity of pathway expression and post-transcriptional regulation leading to reduced tumor-wise RNA:protein correlations. Tumor-wise RNA:protein correlation could serve as a global index of tumor cellular heterogeneity to identify biomarkers for different tranches of cellular heterogeneity.

The APOLLO cohort's thorough clinical follow up data and comprehensive proteogenomic data provided a unique opportunity to identify biomarkers for LUAD clinical outcome. Strikingly, we found that survival proteins outnumber survival RNAs and that expression of survival RNA-proteins were more correlated than survival RNAs or survival proteins. These results indicate that protein expression has a greater relationship with patient survival compared to RNA. Supporting this phenomenon in a different tumor type, a recent report in gastric carcinoma found greater gene-wise RNA:protein correlation in RNA survival genes than in non-survival genes (Mun et al., 2019). RNA and protein expression signatures based on these gene sets, including survival RNAs, survival proteins, and survival RNA-proteins, significantly predicted survival outcomes in both the APOLLO and in an independent validation cohort. Therefore, proteomic expression signatures can reproducibly predict patient outcome in LUAD across cohorts, similar to RNA expression signatures that have been reported previously. Protein expression markers may have superior performance in clinically-available FFPE specimens and small volume biopsies (Hood et al., 2005).

Finally, our proteogenomic analysis ascribed new features to the LUAD expression subtypes, including a concentration of immune features in the PI subtype-interferon-γ signaling, PD-L1 expression, and high TMB. PI subtype might offer unique predictive capacity as a unitive biomarker for the LUADs most responsive to immunotherapy, rather than a threshold on a single analytes such as PD-L1 expression (Herbst et al., 2016) or TMB (Marabelle et al., 2020). In contrast, our integrative analysis found that TRU harbors EGFR signaling and kinase activity from PRKCE and RPS6KA1, which may be potential therapeutic targets for TRU tumors. Our integrative analysis also nominated possible therapies for the PP subtype, including CDK inhibitors and glutaminase inhibitors. In summary, our results provide new and significant proteogenomic information on LUAD that is clinically-relevant. Taken together, our comprehensive results may lead to advances in LUAD precision medicine, such as molecularly-informed clinical trials or improved molecular diagnostics.

### Example 2. Aggressive Lung Cancer-Related Molecules as Molecular Predictors of Lung Adenocarcinoma Survival

Using the method described in Example 1, 66 genes were identified as aggressive lung cancer-related molecules (Table 1). Based on empirical distribution, top 7 positive genes and top 7 negative genes were selected from these 66 genes as “top performers” of aggressive lung cancer-related molecules to generate a shorter gene list to facilitate the applications of these aggressive lung cancer-related molecules in, for example, predicting LUAD survival. These fourteen “top performers” (Table 2) were then used to calculate survival scores (overall survival and metastasis-free survival) associated with each subject in the APOLLO cohort and the CPTAC validation cohort.

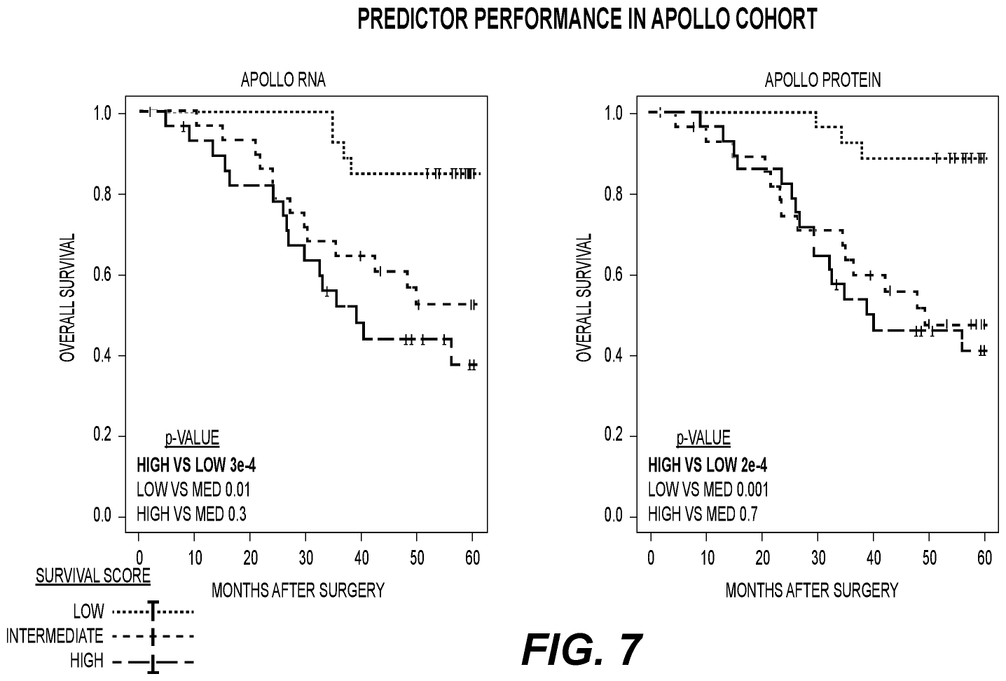

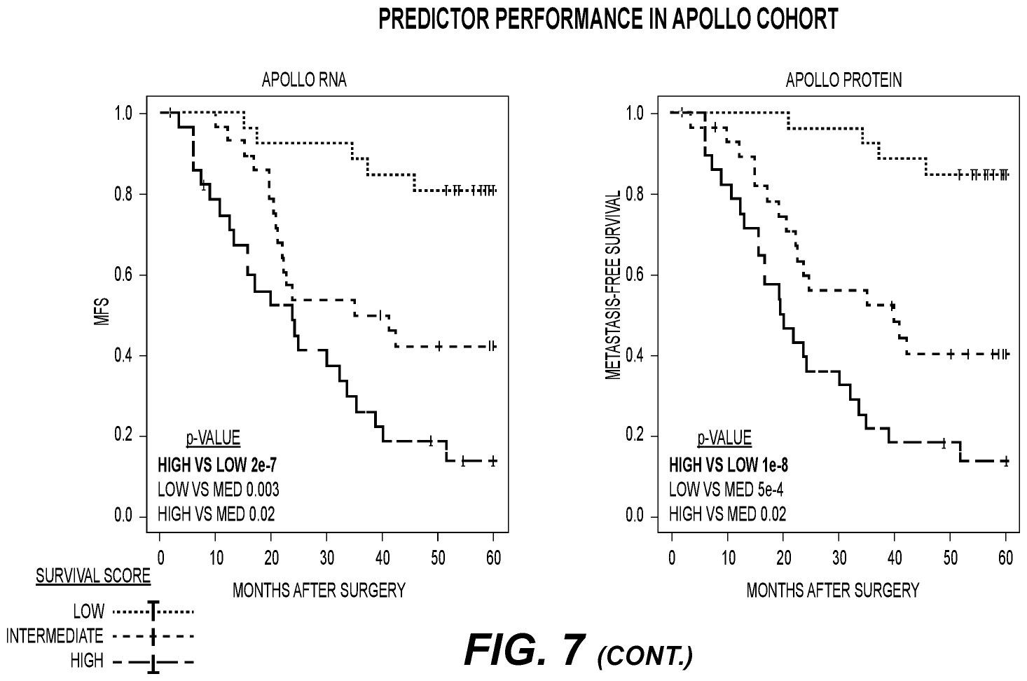

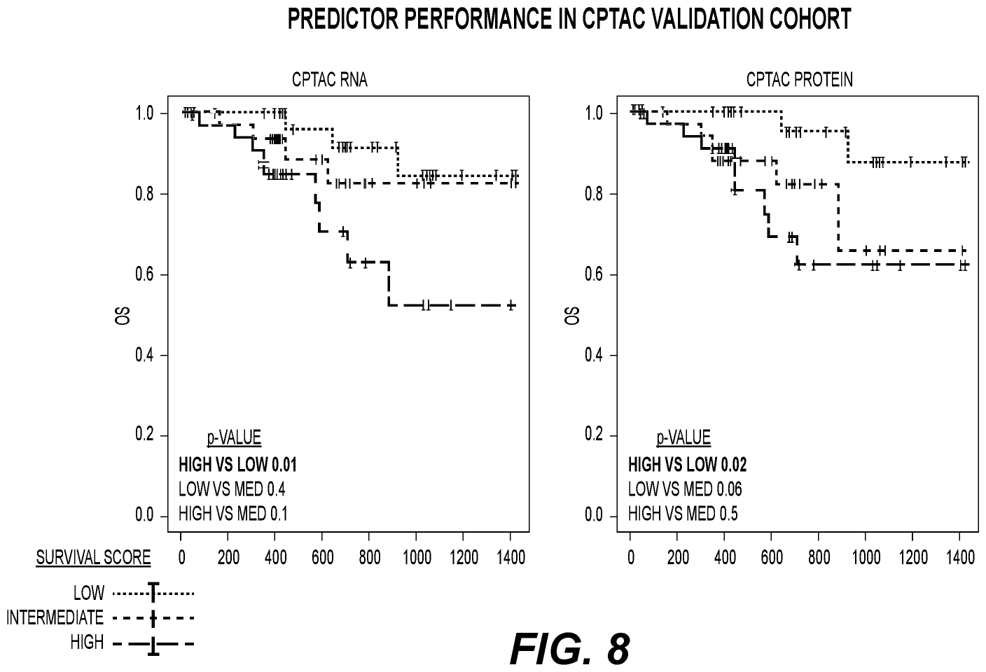

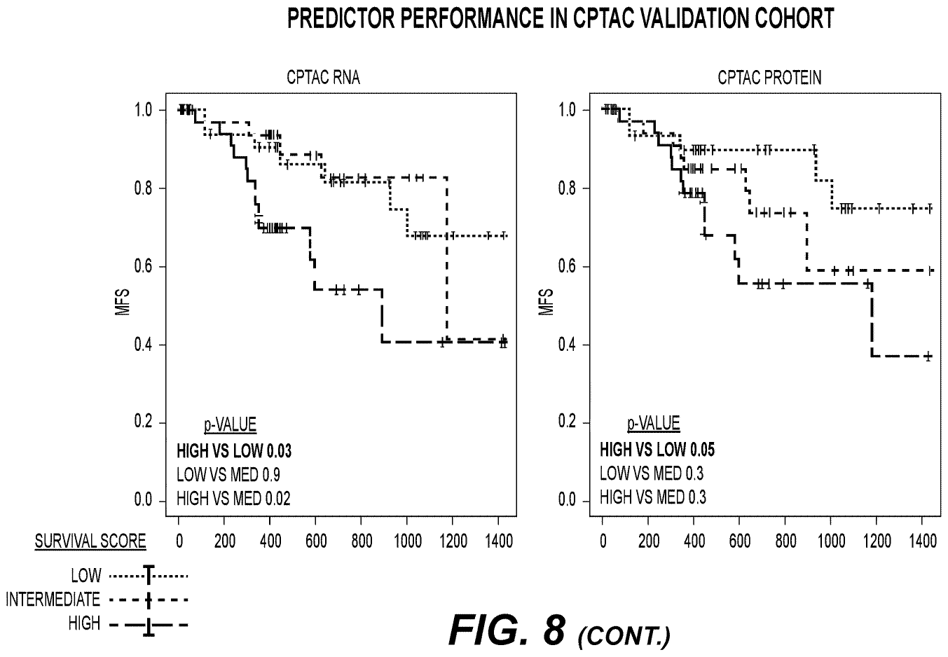

As shown in FIG. 7 and FIG. 8, based on the expression levels of these 14 aggressive lung cancer-related molecules, both the APOLLO cohort (FIG. 7) and the CPTAC validation cohort (FIG. 8) can be divided into three risk groups (low, intermediate, or high) representing three tertiles. Survival outcomes were compared between pairs of risk groups by log ranks tests (p-value).

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. In general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

1-38. (canceled)

39. A method of determining the relative expression levels of a plurality of genes selected from the group consisting of: CLIP1, AVEN, SRPRA, PUS1, MYO1E, KIF26B, FOSL2, MATR3, RPS6KA5, TOR1AIP1, MTX3, UTRN, TMX4, and MCCC1, which comprises
obtaining a sample from a subject; and
measuring the expression levels of each gene in the plurality relative to a given reference or control.

40. The method according to claim 39, wherein the sample is a lung tissue sample.

41. The method according to claim 39, wherein the sample is a tumor sample.

42. The method according to claim 39, wherein the subject has or is at risk of having a lung cancer.

43. The method according to claim 42, wherein the lung cancer is lung adenocarcinoma.

44. The method according to claim 43, which further comprises identifying the molecular subtype of the lung adenocarcinoma.

45. The method according to claim 39, wherein the expression levels are measured based on protein expression and/or RNA expression.

46. The method according to claim 39, wherein the plurality of genes consists of CLIP1, AVEN, SRPRA, PUS1, MYO1E, KIF26B, FOSL2, MATR3, RPS6KA5, TOR1AIP1, MTX3, UTRN, TMX4, and MCCC1.

47. A method of treating a subject who has or is at risk of having a lung cancer, which comprises
determining the expression levels of a plurality of genes selected from the group consisting of: CLIP1, AVEN, SRPRA, PUS1, MYO1E, KIF26B, FOSL2, MATR3, RPS6KA5, TOR1AIP1, MTX3, UTRN, TMX4, and MCCC1 in a sample obtained from the subject;
where the expression levels of one or more of CLIP1, AVEN, SRPRA, PUS1, MYO1E, KIF26B, and FOSL2 are up-regulated and the expression levels of one or more of MATR3, RPS6KA5, TOR1AIP1, MTX3, UTRN, TMX4, and MCCC1 are down-regulated, treating the subject for having or being at risk of having an aggressive form of lung cancer by administering to the subject a chemoprevention treatment or increasing the amount of the a chemoprevention treatment being administered to the subject.

48. The method according to claim 47, which further comprises determining whether the lung cancer is lung adenocarcinoma.

49. The method according to claim 48, if the lung cancer is lung adenocarcinoma, the method further comprises determining the molecular subtype of the lung adenocarcinoma.

50. The method according to claim 49, which further comprises
a) administering a therapeutically effective amount of one or more immunotherapeutic treatments if the molecular subtype of the lung adenocarcinoma is a proximal-inflammatory (PI) subtype;
b) administering a therapeutically effective amount of one or more inhibitory compounds targeting epidermal growth factor receptor (EGFR) signaling and/or kinase activity from protein kinase C epsilon (PRKCE) and/or ribosomal protein S6 kinase A1 (RPS6KA1) if the molecular subtype of the lung adenocarcinoma is a terminal respiratory unit (TRU) subtype; or
c) administering a therapeutically effective amount of one or more cyclin-dependent kinase (CDK) inhibitors and/or glutaminase inhibitors if the molecular subtype of the lung adenocarcinoma is a proximal-proliferative (PP) subtype.

51. The method according to claim 30, wherein the PI subtype of the lung adenocarcinoma is identified by overexpression of the immune cell markers cluster of differentiation 163 (CD163) and/or vascular cell adhesion protein 1 (VCAM1), the TRU subtype is identified by overexpression of surfactant protein C (SFTPC) and/or thyroid transcription factor 1 (NKX2-1 or TTF1), and the PP subtype of the lung adenocarcinoma is identified by overexpression of thymine DNA glycosylase (TDG) and/or glutathione peroxidase 2 (GPX2).

52. The method according to claim 47, wherein the plurality of genes consist of CLIP1, AVEN, SRPRA, PUS1, MYO1E, KIF26B, FOSL2, MATR3, RPS6KA5, TOR1AIP1, MTX3, UTRN, TMX4, and MCCC1.
